(19) United States
(12) Patent Application Publication
(10) Pub. No.: US 2024/0229025 A1
(43) Pub. Date: Jul. 11, 2024

(54) siRNA AND USE THEREOF

(71) Applicant: Ohara Pharmaceutical Co., Ltd., Koka-shi Shiga (JP)

(72) Inventors: Naoki Morita, Koka-shi Shiga (JP); Yoshinobu Yamamoto, Koka-shi Shiga (JP); Kazuki Miyazaki, Koka-shi Shiga (JP); Sumire Okada, Koka-shi Shiga (JP)

(21) Appl. No.: 18/022,630

(22) PCT Filed: Sep. 7, 2021
(86) PCT No.: PCT/JP2021/032871

§ 371 Date: Feb. 22, 2023

(30) Foreign Application Priority Data
Sep. 9, 2020 (JP) 2020-150917

(51) Int. Cl. C12N 15/113

(52) U.S. Cl. CPC C12N 15/113; C12N 2310/14; C12N 2310/315; C12N 2310/321; C12N 2310/322; C12N 2320/10

(57) ABSTRACT

The present invention provides siRNA which selectively exhibits silencing of FUS P525L mutation, pharmaceutical compositions comprising the siRNA and therapeutic uses of ALS, as well as methods for screening the ALS therapeutic agents.

### TECHNICAL FIELD

The present invention relates to siRNA which selectively exhibits silencing of FUS P525L mutation, pharmaceutical compositions comprising the siRNA and therapeutic uses of ALS, as well as methods for screening the ALS therapeutic agents.

### TECHNICAL BACKGROUND

Amyotrophic lateral sclerosis (ALS), also known as Lou Gehrig's disease, is the most fatal progressive neurodegenerative disease characterized by the dominant loss of motor neurons (MN) in the primary motor cortex, brainstem, and spinal cord. The loss of motor neurons disrupts basic basal movements such as breathing and typically causes death of the patients within 2 to 5 years after diagnosis. The progression degradation of the patient's motor function severely reduces respiratory ability, requiring some kinds of breathing assistance for survival of the patients. Other symptoms also include muscle weakness in the hands, arms, legs or swallowing muscles, and frontotemporal dementia may also develop in some patients (for example, FTD-ALS). The aerobic age of ALS is between 50 and 70 years old. Therefore, ALS is a common disease among the elderly.

ALS can be classified into two large types, sporadic ALS and familial ALS. Most of ALS are non-hereditary sporadic ALS. Familial ALS is a disease with a relatively small number of patients, accounting for about 5 to 10% of all ALS.

The cause of the onset of ALS is complex. In general, the onset is believed to be a complex genetic disease by mutation of multiple genes coupled with environmental exposure. Ten or more causative genes have been identified, including SOD-1 (Cu2+/Zn2+superoxide dismutase), TDP-43 (TAR DNA binding protein-43 kD), FUS (fused in sarcoma), ANG (angiogenin), ATXN2 (ataxin-2), VCP (valosin-containing protein), OPTN (optineurin), and C9orf72 (chromosome 9 open reading frame 72). However, the exact mechanism of motor neuron degeneration remains unclear.

FUS is known as a causative gene with a high frequency next to SOD1 in familial ALS. FUS, a causative gene of ALS6 linked to chromosome 16, is an RNA binding protein that is identified by Robert et al. of US in 2009, and is known to be a causative gene that is relatively common among young people among familial ALS.

FUS shuttles between the nucleus and cytoplasm and is responsible for important RNA metabolic functions such as DNA repair and splicing regulation. Mutation in this FUS is known to cause abnormal aggregation in the cytoplasm, and the following two hypotheses have been proposed as onset factors of familial ALS. The first one is loss of function in which RNA metabolism to be performed in the nucleus cannot be performed normally, and the second one is toxicity acquisition due to the aggregation of the mutant protein in the cytoplasm.

At the C-terminus of the FUS protein, there is a nuclear localization signal, and mutation in this region may result in a decrease in affinity with transportin, a nuclear transport receptor. In that case, it is believed that the FUS nuclear transfer cannot be performed normally and the mutant FUS accumulates in the cytoplasm. The mutation location of FUS has been actually investigated, and it has been found that many mutations occur in nuclear localization signal sites, such as P495X, G507D, K510R/E, S513P, R514G/S, R514S, G515C, H517Q/P, R518G/K, R521G/C/H, R522G, R524W/T/S, P525L. Moreover, among the mutations within the nuclear localization signal site, the P525L mutation is common in juvenile ALS that develops at age of 10s to 20s. In addition, most of those patients are known to die within two years after onset. However, currently no therapeutic agents have been developed.

It has been reported that wild-type FUS has important RNA metabolic functions as described above, and in fact, the FUS knockout in mouse forebrain cortical neurons results in decreased interaction with the RNA splicing factor SFPQ and change in tau isoforms. Thus, high selectivity to mutant FUS is essential for the development of therapeutic agents for ALS caused by FUS mutations.

On the other hand, examples of reports on siRNA targeting genes with point mutations include patent literature 1 on siRNA targeting EGFR G356D mutation, non-patent literature 1 on siRNA targeting APP V337M mutation, and non-patent literature 2 on siRNA targeting SOD1 G85R mutation. However, there is no teaching or suggestion on siRNA that selectively exhibits silencing of FUS P525L mutation.

### PRIOR ART LITERATURES

### Patent Literature

1. U.S. Pat. No. 5,941,842

### Non-Patent Literatures

1. Nucleic Acids Research, Miller V M. et al. p 661—668: 2004; 2. Aging Cell, Ding H, p 209—217: 2003.

### SUMMARY OF THE INVENTION

### Problems to be Solved by the Invention

It is an object of the present invention to provide siRNA that selectively exhibits silencing of FUS P525L mutation. It is a further object of the present invention to provide pharmaceutical compositions comprising the siRNA of the present invention, and therapeutic agents for ALS, as well as methods for screening ALS therapeutic agents.

### Means for Solving the Problems

The inventors focused on FUS P525L mutation, designed the siRNA against mutant FUS mRNAs comprising the P525L mutation portion, and further found out the siRNA that highly selectively exhibits mutant mRNA silencing from among some sequences thereof. In other words, the inventors have found that ALS can be treated with a pharmaceutical composition comprising the siRNA that highly selectively exhibits mutant mRNA silencing, and have completed the present invention.

That is, the objects of the present invention are achieved by the following inventions.
[1] An siRNA comprising a sense strand and an antisense strand, wherein the antisense strand comprises a region complementary or substantially complementary to a portion of the mRNA encoding FUS P525L mutation, and wherein the complementary region is 19 to 21 nucleotides in length.
[2] The siRNA of [1], where the antisense strand comprises a sequence identical or substantially identical to SEQ ID NO: 2, SEQ ID NO: 4, SEQ ID NO: 6, SEQ ID NO: 8, SEQ ID NO: 10, SEQ ID NO: 12, SEQ ID NO: 16, SEQ ID NO: 18, SEQ ID NO: 20, SEQ ID NO: 22, SEQ ID NO: 24, SEQ ID NO: 28, or SEQ ID NO: 32.
[3] The siRNA of [1], wherein the antisense strand comprises a sequence identical or substantially identical to SEQ ID NO: 2, SEQ ID NO: 4, SEQ ID NO: 6, SEQ ID NO: 8, SEQ ID NO: 10, SEQ ID NO: 12, SEQ ID NO: 16, SEQ ID NO: 18, SEQ ID NO: 20, SEQ ID NO: 22, or SEQ ID NO: 32.
[4] The siRNA of [1], wherein the antisense strand comprises a sequence identical or substantially identical to SEQ ID NO: 2, SEQ ID NO: 4, SEQ ID NO: 6, SEQ ID NO: 8, SEQ ID NO: 12, SEQ ID NO: 16, SEQ ID NO: 18, SEQ ID NO: 20, SEQ ID NO: 22, SEQ ID NO: 24, SEQ ID NO: 28, or SEQ ID NO: 32.
[5] The siRNA of [1], wherein the antisense strand comprises a sequence identical or substantially identical to SEQ ID NO: 4, SEQ ID NO: 6, SEQ ID NO: 12, SEQ ID NO: 16, SEQ ID NO: 18, SEQ ID NO: 20, or SEQ ID NO: 22.
[6] The siRNA of [1], comprising a sequence identical or substantially identical to
double-stranded RNA consisting of the sense strand of SEQ ID NO: 1 and the antisense strand of SEQ ID NO: 2,
double-stranded RNA consisting of the sense strand of SEQ ID NO: 3 and the antisense strand of SEQ ID NO: 4,
double-stranded RNA consisting of the sense strand of SEQ ID NO: 5 and the antisense strand of SEQ ID NO: 6,
double-stranded RNA consisting of the sense strand of SEQ ID NO: 7 and the antisense strand of SEQ ID NO: 8,
double-stranded RNA consisting of the sense strand of SEQ ID NO: 9 and the antisense strand of SEQ ID NO: 10,
double-stranded RNA consisting of the sense strand of SEQ ID NO: 11 and the antisense strand of SEQ ID NO: 12,
double-stranded RNA consisting of the sense strand of SEQ ID NO: 15 and the antisense strand of SEQ ID NO: 16,
double-stranded RNA consisting of the sense strand of SEQ ID NO: 17 and the antisense strand of SEQ ID NO: 18,
double-stranded RNA consisting of the sense strand of SEQ ID NO: 19 and the antisense strand of SEQ ID NO: 20,
double-stranded RNA consisting of the sense strand of SEQ ID NO: 21 and the antisense strand of SEQ ID NO: 22,
double-stranded RNA consisting of the sense strand of SEQ ID NO: 23 and the antisense strand of SEQ ID NO: 24,
double-stranded RNA consisting of the sense strand of SEQ ID NO: 27 and the antisense strand of SEQ ID NO: 28,
double-stranded RNA consisting of the sense strand of SEQ ID NO: 31 and the antisense strand of SEQ ID NO: 32,
double-stranded RNA consisting of the sense strand of SEQ ID NO: 39 and the antisense strand of SEQ ID NO: 40,
double-stranded RNA consisting of the sense strand of SEQ ID NO: 41 and the antisense strand of SEQ ID NO: 42,
double-stranded RNA consisting of the sense strand of SEQ ID NO: 43 and the antisense strand of SEQ ID NO: 44,
double-stranded RNA consisting of the sense strand of SEQ ID NO: 45 and the antisense strand of SEQ ID NO: 46,
double-stranded RNA consisting of the sense strand of SEQ ID NO: 47 and the antisense strand of SEQ ID NO: 48,
double-stranded RNA consisting of the sense strand of SEQ ID NO: 49 and the antisense strand of SEQ ID NO: 50,
double-stranded RNA consisting of the sense strand of SEQ ID NO: 53 and the antisense strand of SEQ ID NO: 54,
double-stranded RNA consisting of the sense strand of SEQ ID NO: 55 and the antisense strand of SEQ ID NO: 56,
double-stranded RNA consisting of the sense strand of SEQ ID NO: 57 and the antisense strand of SEQ ID NO: 58,
double-stranded RNA consisting of the sense strand of SEQ ID NO: 59 and the antisense strand of SEQ ID NO: 60,
double-stranded RNA consisting of the sense strand of SEQ ID NO: 61 and the antisense strand of SEQ ID NO: 62,
double-stranded RNA consisting of the sense strand of SEQ ID NO: 71 and the antisense strand of SEQ ID NO: 72,
double-stranded RNA consisting of the sense strand of SEQ ID NO: 89 and the antisense strand of SEQ ID NO: 90,
double-stranded RNA consisting of the sense strand of SEQ ID NO: 95 and the antisense strand of SEQ ID NO: 96,
double-stranded RNA composed of the sense strand of SEQ ID NO: 97 and the antisense strand of SEQ ID NO: 98, or
double-stranded RNA consisting of the sense strand of SEQ ID NO: 113 and the antisense strand of SEQ ID NO: 114.
[7] The siRNA of [1], comprising a sequence identical or substantially identical to double-stranded RNA consisting of the sense strand of SEQ ID NO: 1 and the antisense strand of SEQ ID NO: 2,
double-stranded RNA consisting of the sense strand of SEQ ID NO: 3 and the antisense strand of SEQ ID NO: 4,
double-stranded RNA consisting of the sense strand of SEQ ID NO: 5 and the antisense strand of SEQ ID NO: 6,
double-stranded RNA consisting of the sense strand of SEQ ID NO: 7 and the antisense strand of SEQ ID NO: 8,
double-stranded RNA consisting of the sense strand of SEQ ID NO: 9 and the antisense strand of SEQ ID NO: 10,
double-stranded RNA consisting of the sense strand of SEQ ID NO: 11 and the antisense strand of SEQ ID NO: 12,
double-stranded RNA consisting of the sense strand of SEQ ID NO: 15 and the antisense strand of SEQ ID NO: 16,
double-stranded RNA consisting of the sense strand of SEQ ID NO: 17 and the antisense strand of SEQ ID NO: 18,
double-stranded RNA consisting of the sense strand of SEQ ID NO: 19 and the antisense strand of SEQ ID NO: 20,
double-stranded RNA consisting of the sense strand of SEQ ID NO: 21 and the antisense strand of SEQ ID NO: 22,
double-stranded RNA consisting of the sense strand of SEQ ID NO: 45 and the antisense strand of SEQ ID NO: 46,
double-stranded RNA consisting of the sense strand of SEQ ID NO: 47 and the antisense strand of SEQ ID NO: 48,
double-stranded RNA consisting of the sense strand of SEQ ID NO: 49 and the antisense strand of SEQ ID NO: 50,
double-stranded RNA consisting of the sense strand of SEQ ID NO: 53 and the antisense strand of SEQ ID NO: 54,
double-stranded RNA consisting of the sense strand of SEQ ID NO: 55 and the antisense strand of SEQ ID NO: 56,
double-stranded RNA consisting of the sense strand of SEQ ID NO: 57 and the antisense strand of SEQ ID NO: 58,
double-stranded RNA consisting of the sense strand of SEQ ID NO: 59 and the antisense strand of SEQ ID NO: 60,
double-stranded RNA consisting of the sense strand of SEQ ID NO: 89 and the antisense strand of SEQ ID NO: 90,
double-stranded RNA consisting of the sense strand of SEQ ID NO: 97 and the antisense strand of SEQ ID NO: 98, or
double-stranded RNA consisting of the sense strand of SEQ ID NO: 113 and the antisense strand of SEQ ID NO: 114.
[8] The siRNA of [1], comprising a sequence identical or substantially identical to
double-stranded RNA consisting of the sense strand of SEQ ID NO: 3 and the antisense strand of SEQ ID NO: 4,
double-stranded RNA consisting of the sense strand of SEQ ID NO: 5 and the antisense strand of SEQ ID NO: 6,
double-stranded RNA consisting of the sense strand of SEQ ID NO: 7 and the antisense strand of SEQ ID NO: 8,
double-stranded RNA consisting of the sense strand of SEQ ID NO: 11 and the antisense strand of SEQ ID NO: 12,
double-stranded RNA consisting of the sense strand of SEQ ID NO: 15 and the antisense strand of SEQ ID NO: 16,
double-stranded RNA consisting of the sense strand of SEQ ID NO: 17 and the antisense strand of SEQ ID NO: 18,
double-stranded RNA consisting of the sense strand of SEQ ID NO: 19 and the antisense strand of SEQ ID NO: 20,
double-stranded RNA consisting of the sense strand of SEQ ID NO: 21 and the antisense strand of SEQ ID NO: 22,
double-stranded RNA consisting of the sense strand of SEQ ID NO: 23 and the antisense strand of SEQ ID NO: 24,
double-stranded RNA consisting of the sense strand of SEQ ID NO: 27 and the antisense strand of SEQ ID NO: 28,
double-stranded RNA consisting of the sense strand of SEQ ID NO: 31 and the antisense strand of SEQ ID NO: 32,
double-stranded RNA consisting of the sense strand of SEQ ID NO: 39 and the antisense strand of SEQ ID NO: 40,
double-stranded RNA consisting of the sense strand of SEQ ID NO: 41 and the antisense strand of SEQ ID NO: 42,
double-stranded RNA consisting of the sense strand of SEQ ID NO: 43 and the antisense strand of SEQ ID NO: 44,
double-stranded RNA consisting of the sense strand of SEQ ID NO: 45 and the antisense strand of SEQ ID NO: 46,
double-stranded RNA consisting of the sense strand of SEQ ID NO: 49 and the antisense strand of SEQ ID NO: 50,
double-stranded RNA consisting of the sense strand of SEQ ID NO: 53 and the antisense strand of SEQ ID NO: 54,
double-stranded RNA consisting of the sense strand of SEQ ID NO: 55 and the antisense strand of SEQ ID NO: 56,
double-stranded RNA consisting of the sense strand of SEQ ID NO: 57 and the antisense strand of SEQ ID NO: 58,
double-stranded RNA consisting of the sense strand of SEQ ID NO: 59 and the antisense strand of SEQ ID NO: 60,
double-stranded RNA consisting of the sense strand of SEQ ID NO: 61 and the antisense strand of SEQ ID NO: 62,
double-stranded RNA consisting of the sense strand of SEQ ID NO: 71 and the antisense strand of SEQ ID NO: 72,
double-stranded RNA consisting of the sense strand of SEQ ID NO: 89 and the antisense strand of SEQ ID NO: 90,
double-stranded RNA consisting of the sense strand of SEQ ID NO: 95 and the antisense strand of SEQ ID NO: 96, or
double-stranded RNA consisting of the sense strand of SEQ ID NO: 97 and the antisense strand of SEQ ID NO: 98.
[9] The siRNA of [1], comprising a sequence identical or substantially identical to
double-stranded RNA consisting of the sense strand of SEQ ID NO: 3 and the antisense strand of SEQ ID NO: 4,
double-stranded RNA consisting of the sense strand of SEQ ID NO: 5 and the antisense strand of SEQ ID NO: 6,
double-stranded RNA consisting of the sense strand of SEQ ID NO: 11 and the antisense strand of SEQ ID NO: 12,
double-stranded RNA consisting of the sense strand of SEQ ID NO: 15 and the antisense strand of SEQ ID NO: 16,
double-stranded RNA consisting of the sense strand of SEQ ID NO: 17 and the antisense strand of SEQ ID NO: 18,
double-stranded RNA consisting of the sense strand of SEQ ID NO: 19 and the antisense strand of SEQ ID NO: 20,
double-stranded RNA consisting of the sense strand of SEQ ID NO: 21 and the antisense strand of SEQ ID NO: 22,
double-stranded RNA consisting of the sense strand of SEQ ID NO: 45 and the antisense strand of SEQ ID NO: 46,
double-stranded RNA consisting of the sense strand of SEQ ID NO: 49 and the antisense strand of SEQ ID NO: 50,
double-stranded RNA consisting of the sense strand of SEQ ID NO: 53 and the antisense strand of SEQ ID NO: 54,
double-stranded RNA consisting of the sense strand of SEQ ID NO: 55 and the antisense strand of SEQ ID NO: 56,
double-stranded RNA consisting of the sense strand of SEQ ID NO: 57 and the antisense strand of SEQ ID NO: 58,
double-stranded RNA consisting of the sense strand of SEQ ID NO: 59 and the antisense strand of SEQ ID NO: 60,
double-stranded RNA consisting of the sense strand of SEQ ID NO: 89 and the antisense strand of SEQ ID NO: 90, or
double-stranded RNA consisting of the sense strand of SEQ ID NO: 97 and the antisense strand of SEQ ID NO: 98.
[10] The siRNA of any of [1] to [9], wherein the siRNA comprises an overhang of 2 nucleotides in length at the 3′ end of its sense strand and/or antisense strand and is a 21 to 23 base pair.
[11] The siRNA of any of [1] to [10], wherein the siRNA comprises at least one modified nucleotide.
[12] The siRNA of any of [1] to [11], wherein the at least one modified nucleotide comprises a 5′—phosphorothioate group.
[13] The siRNA of any of [1] to [12], wherein the at least one modified nucleotide is selected from the group consisting of 2′-deoxy-modified nucleotide, 2′-deoxy-2′-fluoro-modified nucleotide, 2′-O-methyl modified nucleotide, 2′-O-methoxyethyl modified nucleotide, and the nucleotide wherein the 2′-O atom and 4′-C atom are crosslinked via a methylene or ethylene group.
[14] The siRNA of any of [1] to [13] for silencing of FUS P525L mutation.

The siRNA of any of [1] to [13] for selectively silencing of FUS P525L mutation without substantially silencing of wild-type FUS.
[16] A pharmaceutical composition comprising the siRNA of any of [1] to [13].
[17] A pharmaceutical composition comprising the siRNA of any of [1] to [13], for silencing of FUS P525L mutation.
[18] A pharmaceutical composition comprising the siRNA of any of [1] to [13], for selectively silencing of FUS P525L mutation without substantially silencing of wild-type FUS.
[19] A FUS P525L mutation expression inhibitor comprising the siRNA of any of [1] to [13].
[20] An ALS therapeutic agent comprising the siRNA of any of [1] to [13], or a pharmaceutical composition of any of [16] to [18].
[21] An ALS therapeutic agent of [20], wherein the ALS is juvenile ALS having FUS P525L mutation.
[22] A method of treating ALS characterized by administering an effective amount of the siRNA of any of [1] to [13].
[23] Use of the siRNA of any of [1] to [13] for producing an ALS therapeutic agent.
[24] A DNA vector for expressing the siRNA of any of [1] to [13] in cells.

A pharmaceutical composition comprising the DNA vector of [24] and a pharmaceutically acceptable carrier.
[26] A Cell comprising the DNA vector of [24].

The cell of [26], wherein the cell is a mammalian cell.

A method of screening an ALS therapeutic agent that selectively exhibits silencing of FUS P525L mutation, characterized by determining a silencing rate of wild-type FUS and a silencing rate of FUS P525L mutation.

A method of screening siRNA for use as an active ingredient of the ALS therapeutic agents, comprising
(1) a step of designing the siRNAs comprising a region complementary or substantially complementary to a portion of mRNA encoding FUS P525L mutation, wherein the complementary region is 19 to 21 nucleotides in length, (2) a step of producing the siRNAs designed in step (1), and
(3) a step of screening for the siRNA that selectively exhibits silencing of FUS P525L mutation without substantially silencing of wild-type FUS, from the siRNAs produced in step (2).
[30] A method of producing an ALS therapeutic agent comprising the siRNA that selectively exhibits silencing of FUS P525L mutation as an active ingredient, comprising
(1) a step of designing the siRNAs comprising a region complementary or substantially complementary to a portion of mRNA encoding FUS P525L mutation, wherein the complementary region is 19 to 21 nucleotides in length,
(2) a step of producing the siRNAs designed in step (1),
(3) a step of screening for the siRNA that selectively exhibits silencing of FUS P525L mutation without substantially silencing of wild-type FUS, from the siRNAs produced in step (2),
(4) a step of producing a pharmaceutical composition containing the siRNA screened in step (3) as an active ingredient, and
(5) a step of confirming the effect of the pharmaceutical composition produced in step (4) as an ALS therapeutic agent.

### Effects of the Invention

By using the siRNA of the present invention, silencing of FUS P525L mutation can be exhibited selectively while the silencing of wild-type FUS is substantially not exhibited. In addition, ALS can be treated by the pharmaceutical composition comprising the siRNA of the present invention.

### BRIEF DESCRIPTIONS OF DRAWINGS

FIG. 1 shows the effect of 21 mer siRNA on the expression rate of FUS P525L mutation.

FIG. 2 shows the effect of 21 mer siRNA on silencing of FUS P525L mutation.

FIG. 3 shows the effect of 22 mer siRNA on the expression rate of FUS P525L mutation.

FIG. 4 shows the effect of 22 mer siRNA on silencing of FUS P525L mutation.

FIG. 5 shows the effect of 23 mer siRNA on the expression rate of FUS P525L mutation.

FIG. 6 shows the effect of 23 mer siRNA on silencing of FUS P525L mutation.

FIG. 7 shows representative images in co-expressed HEK293 cell lines.

FIG. 8 shows the effect of 21 mer siRNA on the expression rate of FUS P525L mutation in co-expressed HEK293 cell lines.

FIG. 9 shows the effect of 21 mer siRNA on silencing of FUS P525L mutation in co-expressed HEK293 cell lines.

FIG. 10 shows the effect of 22 mer siRNA on the expression rate of FUS P525L mutation in co-expressed HEK293 cell lines.

FIG. 11 shows the effect of 22 mer siRNA on silencing of FUS P525L mutation in co-expressed HEK293 cell lines.

FIG. 12 shows the effect of 23 mer siRNA on the expression rate of FUS P525L mutation in co-expressed HEK293 cell lines.

FIG. 13 shows the effect of 23 mer siRNA on silencing of FUS P525L mutation in co-expressed HEK293 cell lines.

FIG. 14 shows the concentration-dependent effect of siRNA-010 on silencing of FUS P525L mutation.

FIG. 15 shows the concentration-dependent effect of siRNA-029 on silencing of FUS P525L mutation.

FIG. 16 shows the concentration-dependent effect of siRNA-049 on silencing of FUS P525L mutation.

FIG. 17 shows the time-dependent effect of siRNA-010 on silencing of FUS P525L mutation.

FIG. 18 shows the time-dependent effect of siRNA-029 on silencing of FUS P525L mutation.

FIG. 19 shows the time-dependent effect of siRNA-049 on silencing of FUS P525L mutation.

### EMBODIMENTS OF THE INVENTION

The siRNA (small interfering RNA) of the present invention is a double-stranded RNA comprised of RNA (antisense strand) complementary to targeted FUS P525L mutation mRNA (transcript) and RNA (sense strand) complementary to the RNA.

In general, when siRNA is transfected into a cell, target gene silencing is promoted via mRNA degradation mediated RNA interference. However, the siRNA of the present invention can target and degrade the mRNA of FUS P525L mutation, and selectively exhibits silencing of FUS P525L mutation involved in ALS.

The siRNA of the present invention comprises an antisense strand complementary or substantially complementary to a portion of the mRNA encoding FUS P525L mutation, wherein the complementary region is 19 to 21 nucleotides in length.

Moreover, in the siRNA of the present invention, each of the sense strand and antisense strand is 19 to 26 nucleotides, preferably 19 to 23 nucleotides in length.

The terms “complementary” and “substantially complementary” herein mean that the opposing bases between the sense strand and the antisense strand of the siRNA or between the antisense strand of the siRNA and the targeted mRNA can form a hydrogen bond, as understood from the context in which they are used.

The siRNA of the present invention comprises an antisense strand having a sequence identical or substantially identical to any of the SEQ ID NOs shown in Table 1 below, preferably however, an antisense strand having a sequence identical or substantially identical to SEQ ID NO: 2, SEQ ID NO: 4, SEQ ID NO: 6, SEQ ID NO: 8, SEQ ID NO: 10, SEQ ID NO: 12, SEQ ID NO: 16, SEQ ID NO: 18, SEQ ID NO: 20, SEQ ID NO: 22, or SEQ ID NO: 32.

TABLE 1
Strand: s = sense, as = antisense

| | | | SEQ | Sequence with 3′- | SEQ |
|---|---|---|---|---|---|
| | | Sequence | ID | dinucleotide overhangs | ID |
| siRNA | Strand | (5′→3′) | NO | (5′→3′) | NO |
| siRNA- | s | AGGAUCGCAGGGAGAGGCU | 1 | AGGAUCGCAGGGAGAGGCUdTdT | 121 |
| 001 | as | AGCCUCUCCCUGCGAUCCU | 2 | AGCCUCUCCCUGCGAUCCUdTdT | 122 |
| siRNA- | s | GGAUCGCAGGGAGAGGCUG | 3 | GGAUCGCAGGGAGAGGCUGdTdT | 123 |
| 002 | as | CAGCCUCUCCCUGCGAUCC | 4 | CAGCCUCUCCCUGCGAUCCdTdT | 124 |
| siRNA- | s | GAUCGCAGGGAGAGGCUGU | 5 | GAUCGCAGGGAGAGGCUGUdTdT | 125 |
| 003 | as | ACAGCCUCUCCCUGCGAUC | 6 | ACAGCCUCUCCCUGCGAUCdTdT | 126 |
| siRNA- | s | AUCGCAGGGAGAGGCUGUA | 7 | AUCGCAGGGAGAGGCUGUAdTdT | 127 |
| 004 | as | UACAGCCUCUCCCUGCGAU | 8 | UACAGCCUCUCCCUGCGAUdTdT | 128 |
| siRNA- | s | UCGCAGGGAGAGGCUGUAU | 9 | UCGCAGGGAGAGGCUGUAUdTdT | 129 |
| 005 | as | AUACAGCCUCUCCCUGCGA | 10 | AUACAGCCUCUCCCUGCGAdTdT | 130 |
| siRNA- | s | CGCAGGGAGAGGCUGUAUU | 11 | CGCAGGGAGAGGCUGUAUUdTdT | 131 |
| 006 | as | AAUACAGCCUCUCCCUGCG | 12 | AAUACAGCCUCUCCCUGCGdTdT | 132 |
| siRNA- | s | GCAGGGAGAGGCUGUAUUA | 13 | GCAGGGAGAGGCUGUAUUAdTdT | 133 |
| 007 | as | UAAUACAGCCUCUCCCUGC | 14 | UAAUACAGCCUCUCCCUGCdTdT | 134 |
| siRNA- | s | CAGGGAGAGGCUGUAUUAA | 15 | CAGGGAGAGGCUGUAUUAAdTdT | 135 |
| 008 | as | UUAAUACAGCCUCUCCCUG | 16 | UUAAUACAGCCUCUCCCUGdTdT | 136 |
| siRNA- | s | AGGGAGAGGCUGUAUUAAU | 17 | AGGGAGAGGCUGUAUUAAUdTdT | 137 |
| 009 | as | AUUAAUACAGCCUCUCCCU | 18 | AUUAAUACAGCCUCUCCCUdTdT | 138 |
| siRNA- | s | GGGAGAGGCUGUAUUAAUU | 19 | GGGAGAGGCUGUAUUAAUUdTdT | 139 |
| 010 | as | AAUUAAUACAGCCUCUCCC | 20 | AAUUAAUACAGCCUCUCCCdTdT | 140 |
| siRNA- | s | GGAGAGGCUGUAUUAAUUA | 21 | GGAGAGGCUGUAUUAAUUAdTdT | 141 |
| 011 | as | UAAUUAAUACAGCCUCUCC | 22 | UAAUUAAUACAGCCUCUCCdTdT | 142 |
| siRNA- | s | GAGAGGCUGUAUUAAUUAG | 23 | GAGAGGCUGUAUUAAUUAGdTdT | 143 |
| 012 | as | CUAAUUAAUACAGCCUCUC | 24 | CUAAUUAAUACAGCCUCUCdTdT | 144 |
| siRNA- | s | AGAGGCUGUAUUAAUUAGC | 25 | AGAGGCUGUAUUAAUUAGCdTdT | 145 |
| 013 | as | GCUAAUUAAUACAGCCUCU | 26 | GCUAAUUAAUACAGCCUCUdTdT | 146 |
| siRNA- | s | GAGGCUGUAUUAAUUAGCC | 27 | GAGGCUGUAUUAAUUAGCCdTdT | 147 |
| 014 | as | GGCUAAUUAAUACAGCCUC | 28 | GGCUAAUUAAUACAGCCUCdTdT | 148 |
| siRNA- | s | AGGCUGUAUUAAUUAGCCU | 29 | AGGCUGUAUUAAUUAGCCUdTdT | 149 |
| 015 | as | AGGCUAAUUAAUACAGCCU | 30 | AGGCUAAUUAAUACAGCCUdTdT | 150 |
| siRNA- | s | GGCUGUAUUAAUUAGCCUG | 31 | GGCUGUAUUAAUUAGCCUGdTdT | 151 |
| 016 | as | CAGGCUAAUUAAUACAGCC | 32 | CAGGCUAAUUAAUACAGCCdTdT | 152 |
| siRNA- | s | GCUGUAUUAAUUAGCCUGG | 33 | GCUGUAUUAAUUAGCCUGGdTdT | 153 |
| 017 | as | CCAGGCUAAUUAAUACAGC | 34 | CCAGGCUAAUUAAUACAGCdTdT | 154 |
| siRNA- | s | CUGUAUUAAUUAGCCUGGC | 35 | CUGUAUUAAUUAGCCUGGCdTdT | 155 |
| 018 | as | GCCAGGCUAAUUAAUACAG | 36 | GCCAGGCUAAUUAAUACAGdTdT | 156 |
| siRNA- | s | UGUAUUAAUUAGCCUGGCU | 37 | UGUAUUAAUUAGCCUGGCUdTdT | 157 |
| 019 | as | AGCCAGGCUAAUUAAUACA | 38 | AGCCAGGCUAAUUAAUACAdTdT | 158 |
| siRNA- | s | CAGGAUCGCAGGGAGAGGCU | 39 | CAGGAUCGCAGGGAGAGGCUdTdT | 159 |
| 020 | as | AGCCUCUCCCUGCGAUCCUG | 40 | AGCCUCUCCCUGCGAUCCUGdTdT | 160 |
| siRNA- | s | AGGAUCGCAGGGAGAGGCUG | 41 | AGGAUCGCAGGGAGAGGCUGdTdT | 161 |
| 021 | as | CAGCCUCUCCCUGCGAUCCU | 42 | CAGCCUCUCCCUGCGAUCCUdTdT | 162 |
| siRNA- | s | GGAUCGCAGGGAGAGGCUGU | 43 | GGAUCGCAGGGAGAGGCUGUdTdT | 163 |
| 022 | as | ACAGCCUCUCCCUGCGAUCC | 44 | ACAGCCUCUCCCUGCGAUCCdTdT | 164 |
| siRNA- | s | GAUCGCAGGGAGAGGCUGUA | 45 | GAUCGCAGGGAGAGGCUGUAdTdT | 165 |
| 023 | as | UACAGCCUCUCCCUGCGAUC | 46 | UACAGCCUCUCCCUGCGAUCdTdT | 166 |
| siRNA- | s | AUCGCAGGGAGAGGCUGUAU | 47 | AUCGCAGGGAGAGGCUGUAUdTdT | 167 |
| 024 | as | AUACAGCCUCUCCCUGCGAU | 48 | AUACAGCCUCUCCCUGCGAUdTdT | 168 |
| siRNA- | s | UCGCAGGGAGAGGCUGUAUU | 49 | UCGCAGGGAGAGGCUGUAUUdTdT | 169 |
| 025 | as | AAUACAGCCUCUCCCUGCGA | 50 | AAUACAGCCUCUCCCUGCGAdTdT | 170 |
| siRNA- | s | CGCAGGGAGAGGCUGUAUUA | 51 | CGCAGGGAGAGGCUGUAUUAdTdT | 171 |
| 026 | as | UAAUACAGCCUCUCCCUGCG | 52 | UAAUACAGCCUCUCCCUGCGdTdT | 172 |
| siRNA- | s | GCAGGGAGAGGCUGUAUUAA | 53 | GCAGGGAGAGGCUGUAUUAAdTdT | 173 |
| 027 | as | UUAAUACAGCCUCUCCCUGC | 54 | UUAAUACAGCCUCUCCCUGCdTdT | 174 |
| siRNA- | s | CAGGGAGAGGCUGUAUUAAU | 55 | CAGGGAGAGGCUGUAUUAAUdTdT | 175 |
| 028 | as | AUUAAUACAGCCUCUCCCUG | 56 | AUUAAUACAGCCUCUCCCUGdTdT | 176 |
| siRNA- | s | AGGGAGAGGCUGUAUUAAUU | 57 | AGGGAGAGGCUGUAUUAAUUdTdT | 177 |
| 029 | as | AAUUAAUACAGCCUCUCCCU | 58 | AAUUAAUACAGCCUCUCCCUdTdT | 178 |
| siRNA- | s | GGGAGAGGCUGUAUUAAUUA | 59 | GGGAGAGGCUGUAUUAAUUAdTdT | 179 |
| 030 | as | UAAUUAAUACAGCCUCUCCC | 60 | UAAUUAAUACAGCCUCUCCCdTdT | 180 |
| siRNA- | s | GGAGAGGCUGUAUUAAUUAG | 61 | GGAGAGGCUGUAUUAAUUAGdTdT | 181 |
| 031 | as | CUAAUUAAUACAGCCUCUCC | 62 | CUAAUUAAUACAGCCUCUCCdTdT | 182 |
| siRNA- | s | GAGAGGCUGUAUUAAUUAGC | 63 | GAGAGGCUGUAUUAAUUAGCdTdT | 183 |
| 032 | as | GCUAAUUAAUACAGCCUCUC | 64 | GCUAAUUAAUACAGCCUCUCdTdT | 184 |
| siRNA- | s | AGAGGCUGUAUUAAUUAGCC | 65 | AGAGGCUGUAUUAAUUAGCCdTdT | 185 |
| 033 | as | GGCUAAUUAAUACAGCCUCU | 66 | GGCUAAUUAAUACAGCCUCUdTdT | 186 |
| siRNA- | s | GAGGCUGUAUUAAUUAGCCU | 67 | GAGGCUGUAUUAAUUAGCCUdTdT | 187 |
| 034 | as | AGGCUAAUUAAUACAGCCUC | 68 | AGGCUAAUUAAUACAGCCUCdTdT | 188 |
| siRNA- | s | AGGCUGUAUUAAUUAGCCUG | 69 | AGGCUGUAUUAAUUAGCCUGdTdT | 189 |
| 035 | as | CAGGCUAAUUAAUACAGCCU | 70 | CAGGCUAAUUAAUACAGCCUdTdT | 190 |
| siRNA- | s | GGCUGUAUUAAUUAGCCUGG | 71 | GGCUGUAUUAAUUAGCCUGGdTdT | 191 |
| 036 | as | CCAGGCUAAUUAAUACAGCC | 72 | CCAGGCUAAUUAAUACAGCCdTdT | 192 |
| siRNA- | s | GCUGUAUUAAUUAGCCUGGC | 73 | GCUGUAUUAAUUAGCCUGGCdTdT | 193 |
| 037 | as | GCCAGGCUAAUUAAUACAGC | 74 | GCCAGGCUAAUUAAUACAGCdTdT | 194 |
| siRNA- | s | CUGUAUUAAUUAGCCUGGCU | 75 | CUGUAUUAAUUAGCCUGGCUdTdT | 195 |
| 038 | as | AGCCAGGCUAAUUAAUACAG | 76 | AGCCAGGCUAAUUAAUACAGdTdT | 196 |
| siRNA- | s | UGUAUUAAUUAGCCUGGCUC | 77 | UGUAUUAAUUAGCCUGGCUCdTdT | 197 |
| 039 | as | GAGCCAGGCUAAUUAAUACA | 78 | GAGCCAGGCUAAUUAAUACAdTdT | 198 |
| siRNA- | s | ACAGGAUCGCAGGGAGAGGCU | 79 | ACAGGAUCGCAGGGAGAGGCUdTdT | 199 |
| 040 | as | AGCCUCUCCCUGCGAUCCUGU | 80 | AGCCUCUCCCUGCGAUCCUGUdTdT | 200 |
| siRNA- | s | CAGGAUCGCAGGGAGAGGCUG | 81 | CAGGAUCGCAGGGAGAGGCUGdTdT | 201 |
| 041 | as | CAGCCUCUCCCUGCGAUCCUG | 82 | CAGCCUCUCCCUGCGAUCCUGdTdT | 202 |
| siRNA- | s | AGGAUCGCAGGGAGAGGCUGU | 83 | AGGAUCGCAGGGAGAGGCUGUdTdT | 203 |
| 042 | as | ACAGCCUCUCCCUGCGAUCCU | 84 | ACAGCCUCUCCCUGCGAUCCUdTdT | 204 |
| siRNA- | s | GGAUCGCAGGGAGAGGCUGUA | 85 | GGAUCGCAGGGAGAGGCUGUAdTdT | 205 |
| 043 | as | UACAGCCUCUCCCUGCGAUCC | 86 | UACAGCCUCUCCCUGCGAUCCdTdT | 206 |
| siRNA- | s | GAUCGCAGGGAGAGGCUGUAU | 87 | GAUCGCAGGGAGAGGCUGUAUdTdT | 207 |
| 044 | as | AUACAGCCUCUCCCUGCGAUC | 88 | AUACAGCCUCUCCCUGCGAUCdTdT | 208 |
| siRNA- | s | AUCGCAGGGAGAGGCUGUAUU | 89 | AUCGCAGGGAGAGGCUGUAUUdTdT | 209 |
| 045 | as | AAUACAGCCUCUCCCUGCGAU | 90 | AAUACAGCCUCUCCCUGCGAUdTdT | 210 |
| siRNA- | s | UCGCAGGGAGAGGCUGUAUUA | 91 | UCGCAGGGAGAGGCUGUAUUAdTdT | 211 |
| 046 | as | UAAUACAGCCUCUCCCUGCGA | 92 | UAAUACAGCCUCUCCCUGCGAdTdT | 212 |
| siRNA- | s | CGCAGGGAGAGGCUGUAUUAA | 93 | CGCAGGGAGAGGCUGUAUUAAdTdT | 213 |
| 047 | as | UUAAUACAGCCUCUCCCUGCG | 94 | UUAAUACAGCCUCUCCCUGCGdTdT | 214 |
| siRNA- | s | GCAGGGAGAGGCUGUAUUAAU | 95 | GCAGGGAGAGGCUGUAUUAAUdTdT | 215 |
| 048 | as | AUUAAUACAGCCUCUCCCUGC | 96 | AUUAAUACAGCCUCUCCCUGCdTdT | 216 |
| siRNA- | s | CAGGGAGAGGCUGUAUUAAUU | 97 | CAGGGAGAGGCUGUAUUAAUUdTdT | 217 |
| 049 | as | AAUUAAUACAGCCUCUCCCUG | 98 | AAUUAAUACAGCCUCUCCCUGdTdT | 218 |
| siRNA- | s | AGGGAGAGGCUGUAUUAAUUA | 99 | AGGGAGAGGCUGUAUUAAUUAdTdT | 219 |
| 050 | as | UAAUUAAUACAGCCUCUCCCU | 100 | UAAUUAAUACAGCCUCUCCCUdTdT | 220 |
| siRNA- | s | GGGAGAGGCUGUAUUAAUUAG | 101 | GGGAGAGGCUGUAUUAAUUAGdTdT | 221 |
| 051 | as | CUAAUUAAUACAGCCUCUCCC | 102 | CUAAUUAAUACAGCCUCUCCCdTdT | 222 |
| siRNA- | s | GGAGAGGCUGUAUUAAUUAGC | 103 | GGAGAGGCUGUAUUAAUUAGCdTdT | 223 |
| 052 | as | GCUAAUUAAUACAGCCUCUCC | 104 | GCUAAUUAAUACAGCCUCUCCdTdT | 224 |
| siRNA- | s | GAGAGGCUGUAUUAAUUAGCC | 105 | GAGAGGCUGUAUUAAUUAGCCdTdT | 225 |
| 053 | as | GGCUAAUUAAUACAGCCUCUC | 106 | GGCUAAUUAAUACAGCCUCUCdTdT | 226 |
| siRNA- | s | AGAGGCUGUAUUAAUUAGCCU | 107 | AGAGGCUGUAUUAAUUAGCCUdTdT | 227 |
| 054 | as | AGGCUAAUUAAUACAGCCUCU | 108 | AGGCUAAUUAAUACAGCCUCUdTdT | 228 |
| siRNA- | s | GAGGCUGUAUUAAUUAGCCUG | 109 | GAGGCUGUAUUAAUUAGCCUGdTdT | 229 |
| 055 | as | CAGGCUAAUUAAUACAGCCUC | 110 | CAGGCUAAUUAAUACAGCCUCdTdT | 230 |
| siRNA- | s | AGGCUGUAUUAAUUAGCCUGG | 111 | AGGCUGUAUUAAUUAGCCUGGdTdT | 231 |
| 056 | as | CCAGGCUAAUUAAUACAGCCU | 112 | CCAGGCUAAUUAAUACAGCCUdTdT | 232 |
| siRNA- | s | GGCUGUAUUAAUUAGCCUGGC | 113 | GGCUGUAUUAAUUAGCCUGGCdTdT | 233 |
| 057 | as | GCCAGGCUAAUUAAUACAGCC | 114 | GCCAGGCUAAUUAAUACAGCCdTdT | 234 |
| siRNA- | s | GCUGUAUUAAUUAGCCUGGCU | 115 | GCUGUAUUAAUUAGCCUGGCUdTdT | 235 |
| 058 | as | AGCCAGGCUAAUUAAUACAGC | 116 | AGCCAGGCUAAUUAAUACAGCdTdT | 236 |
| siRNA- | s | CUGUAUUAAUUAGCCUGGCUC | 117 | CUGUAUUAAUUAGCCUGGCUCdTdT | 237 |
| 059 | as | GAGCCAGGCUAAUUAAUACAG | 118 | GAGCCAGGCUAAUUAAUACAGdTdT | 238 |
| siRNA- | s | UGUAUUAAUUAGCCUGGCUCC | 119 | UGUAUUAAUUAGCCUGGCUCCdTdT | 239 |
| 060 | as | GGAGCCAGGCUAAUUAAUACA | 120 | GGAGCCAGGCUAAUUAAUACAdTdT | 240 |

The siRNA of the present invention comprises a sense strand and an antisense strand having the sequences identical or substantially identical to the SEQ ID NOs shown in Table 1 above, preferably however, the sequences identical or substantially identical to
double-stranded RNA comprising a sense strand of SEQ ID NO: 1 and an antisense strand of SEQ ID NO: 2,
double-stranded RNA comprising the sense strand of SEQ ID NO: 3 and the antisense strand of SEQ ID NO: 4,
double-stranded RNA comprising the sense strand of SEQ ID NO: 5 and the antisense strand of SEQ ID NO: 6,
double-stranded RNA comprising the sense strand of SEQ ID NO: 7 and the antisense strand of SEQ ID NO: 8,
double-stranded RNA comprising the sense strand of SEQ ID NO: 9 and the antisense strand of SEQ ID NO: 10,
double-stranded RNA comprising the sense strand of SEQ ID NO: 11 and the antisense strand of SEQ ID NO: 12,
double-stranded RNA comprising the sense strand of SEQ ID NO: 15 and the antisense strand of SEQ ID NO: 16,
double-stranded RNA comprising the sense strand of SEQ ID NO: 17 and the antisense strand of SEQ ID NO: 18,
double-stranded RNA comprising the sense strand of SEQ ID NO: 19 and the antisense strand of SEQ ID NO: 20,
double-stranded RNA comprising the sense strand of SEQ ID NO: 21 and the antisense strand of SEQ ID NO: 22,
double-stranded RNA comprising the sense strand of SEQ ID NO: 23 and the antisense strand of SEQ ID NO: 24,
double-stranded RNA comprising the sense strand of SEQ ID NO: 27 and the antisense strand of SEQ ID NO: 28,
double-stranded RNA comprising the sense strand of SEQ ID NO: 31 and the antisense strand of SEQ ID NO: 32,
double-stranded RNA comprising the sense strand of SEQ ID NO: 39 and the antisense strand of SEQ ID NO: 40,
double-stranded RNA comprising the sense strand of SEQ ID NO: 41 and the antisense strand of SEQ ID NO: 42,
double-stranded RNA comprising the sense strand of SEQ ID NO: 43 and the antisense strand of SEQ ID NO: 44,
double-stranded RNA comprising the sense strand of SEQ ID NO: 45 and the antisense strand of SEQ ID NO: 46,
double-stranded RNA comprising the sense strand of SEQ ID NO: 47 and the antisense strand of SEQ ID NO: 48,
double-stranded RNA comprising the sense strand of SEQ ID NO: 49 and the antisense strand of SEQ ID NO: 50,
double-stranded RNA comprising the sense strand of SEQ ID NO: 53 and the antisense strand of SEQ ID NO: 54,
double-stranded RNA comprising the sense strand of SEQ ID NO: 55 and the antisense strand of SEQ ID NO: 56,
double-stranded RNA comprising the sense strand of SEQ ID NO: 57 and the antisense strand of SEQ ID NO: 58,
double-stranded RNA comprising the sense strand of SEQ ID NO: 59 and the antisense strand of SEQ ID NO: 60,
double-stranded RNA comprising the sense strand of SEQ ID NO: 61 and the antisense strand of SEQ ID NO: 62,
double-stranded RNA comprising the sense strand of SEQ ID NO: 71 and the antisense strand of SEQ ID NO: 72,
double-stranded RNA comprising the sense strand of SEQ ID NO: 89 and the antisense strand of SEQ ID NO: 90,
double-stranded RNA comprising the sense strand of SEQ ID NO: 95 and the antisense strand of SEQ ID NO: 96,
double-stranded RNA composed of the sense strand of SEQ ID NO: 97 and the antisense strand of SEQ ID NO: 98, or
double-stranded RNA comprising the sense strand of SEQ ID NO: 113 and the antisense strand of SEQ ID NO: 114, and more preferably, the sequences identical or substantially identical to
double-stranded RNA comprising a sense strand of SEQ ID NO: 1 and an antisense strand of SEQ ID NO: 2,
double-stranded RNA comprising the sense strand of SEQ ID NO: 3 and the antisense strand of SEQ ID NO: 4,
double-stranded RNA comprising the sense strand of SEQ ID NO: 5 and the antisense strand of SEQ ID NO: 6,
double-stranded RNA comprising the sense strand of SEQ ID NO: 7 and the antisense strand of SEQ ID NO: 8,
double-stranded RNA comprising the sense strand of SEQ ID NO: 9 and the antisense strand of SEQ ID NO: 10,
double-stranded RNA comprising the sense strand of SEQ ID NO: 11 and the antisense strand of SEQ ID NO: 12,
double-stranded RNA comprising the sense strand of SEQ ID NO: 15 and the antisense strand of SEQ ID NO: 16,
double-stranded RNA comprising the sense strand of SEQ ID NO: 17 and the antisense strand of SEQ ID NO: 18,
double-stranded RNA comprising the sense strand of SEQ ID NO: 19 and the antisense strand of SEQ ID NO: 20,
double-stranded RNA comprising the sense strand of SEQ ID NO: 21 and the antisense strand of SEQ ID NO: 22,
double-stranded RNA comprising the sense strand of SEQ ID NO: 45 and the antisense strand of SEQ ID NO: 46,
double-stranded RNA comprising the sense strand of SEQ ID NO: 47 and the antisense strand of SEQ ID NO: 48,
double-stranded RNA comprising the sense strand of SEQ ID NO: 49 and the antisense strand of SEQ ID NO: 50,
double-stranded RNA comprising the sense strand of SEQ ID NO: 53 and the antisense strand of SEQ ID NO: 54,
double-stranded RNA comprising the sense strand of SEQ ID NO: 55 and the antisense strand of SEQ ID NO: 56,
double-stranded RNA comprising the sense strand of SEQ ID NO: 57 and the antisense strand of SEQ ID NO: 58,
double-stranded RNA comprising the sense strand of SEQ ID NO: 59 and the antisense strand of SEQ ID NO: 60,
double-stranded RNA comprising the sense strand of SEQ ID NO: 89 and the antisense strand of SEQ ID NO: 90,
double-stranded RNA composed of the sense strand of SEQ ID NO: 97 and the antisense strand of SEQ ID NO: 98, or
double-stranded RNA comprising the sense strand of SEQ ID NO: 113 and the antisense strand of SEQ ID NO: 114.

As used herein, “substantially identical sequences” means that the sequence numbers in Table 1 may comprise chemical modifications and mismatched bases, so long as the antisense strand of the siRNA and the targeted mRNA retain the ability to form double-stranded RNA, wherein preferably up to 3 mismatched bases, and more preferably up to 1 mismatched base may be comprised.

The sense strand and antisense strand of the siRNA of the present invention may comprise a dinucleotide overhang at the 3′ end. “dT” means deoxythymidine. Overhangs are selected from any of DNA or RNA. For example, dTdT and UU are frequently used. Typically, less expensive dTdT is used.

As used herein, a “nucleotide overhang” refers to a nucleotide that protrudes from a double stranded structure of a siRNA when the 3′ end of a single strand of a non-paired nucleotide or siRNA extends beyond the 5′ end of the other strand, or vice versa.

The siRNA of the present invention may comprise at least one modified nucleotide, wherein at least one of the modified nucleotides may comprise a 5′-phosphorothioate group.

In addition, at least one of the modified nucleotides is selected from the group consisting of 2′-deoxy-modified nucleotides, 2′-deoxy-2′-fluoro-modified nucleotides, 2′-O-methyl modified nucleotides, 2′-O-methoxyethyl modified nucleotides, and nucleotides wherein the 2′-O atoms and 4′-C atoms are crosslinked via methylene or ethylene groups.

As used herein, the term “antisense strand” refers to a strand of a siRNA comprising a region substantially complementary to a targeted sequence. As used herein, the term “complementary region” refers to a region of an antisense strand substantially complementary to a sequence, for example, a targeted sequence defined herein.

As used herein, the term “sense strand” refers to a strand of a siRNA comprising a region substantially complementary to the region of an antisense strand.

The siRNA of the present invention is obtained by selecting and preparing targeted sequences based on FUS P525L mutation mRNA.

For example, the sequence of successive regions of FUS P525L mutation mRNA is selected. Specifically, a mRNA sequence comprising a point mutation and being of 19 to 21 nucleotides before and after the point mutation is selected.

The siRNA of the present invention can be prepared according to the synthetic methods of nucleic acid molecules known per se. Examples of known methods include those described in (i) and (ii) below.
(i) “Creation of Nucleic Acid Drugs and Application”, CMC Publication, 2016
(ii) “Synthetic Technology of Peptide, Nucleic Acid, Sugar Chain Contributing to Research of Middle Molecule Drugs”, CMC Publication, 2018

The siRNA of the present invention can be produced by those skilled in the art, as appropriate, based on the base sequences disclosed herein. Specifically, the double-stranded RNA of the present invention can be produced based on the base sequences described in any of SEQ ID NOs: 1 to 240. If one strand (for example, the base sequence described in SEQ ID NO: 1) is known, those skilled in the art can readily know the base sequence of the other strand (a complementary strand). The siRNA of the present invention can be produced by those skilled in the art, as appropriate, using a commercially available nucleic acid synthesizer. Besides, regarding the synthesis of the desired RNA, a general consignment service for synthesis is available.

The siRNA of the present invention can be synthesized by annealing complementary single-stranded oligonucleotides. Each oligonucleotide can be synthesized according to solid phase synthetic methods using a commercially available amidite. The solid phase synthesis is carried out using a commercially available nucleic acid synthesizer and a solid-phase support. The 3′-end of a nucleotide of a monomer is bound to the surface of solid-phase support via an alkyl chain, and amidite is added. That is, by extending nucleotide one by one from the 3′-end to the 5′-end of the oligonucleotide sequence of interest, an oligonucleotide can be synthesized. A single-stranded RNA of interest can be prepared by cleaving the oligonucleotide from the solid-phase support after completion of the synthetic cycle and then deprotecting the base moiety and 2′ position.

It should be noted that the sequence of the resulting siRNA may have one or several substitutions, deletions, insertions and/or additions to the sequence, if it is possible to induce RNA interference and degrade the targeted mRNA.

The siRNA of the present invention can induce RNA interference, degrade the mRNA of the FUS P525L mutation as a target, and selectively exhibit silencing of FUS P525L mutation involved in ALS.

The selectivity is calculated as follows:

$$Selectivity ( 1 ) = ( the expression rate of wild - type FUS ) \text{} / ( the expression rate of FUS P 525 L mutation )$$

$$or ,$$

$$Selectivity ( 2 ) = ( the silencing rate of FUS P 525 L mutation ) - ( the silencing rate of wild - type FUS )$$

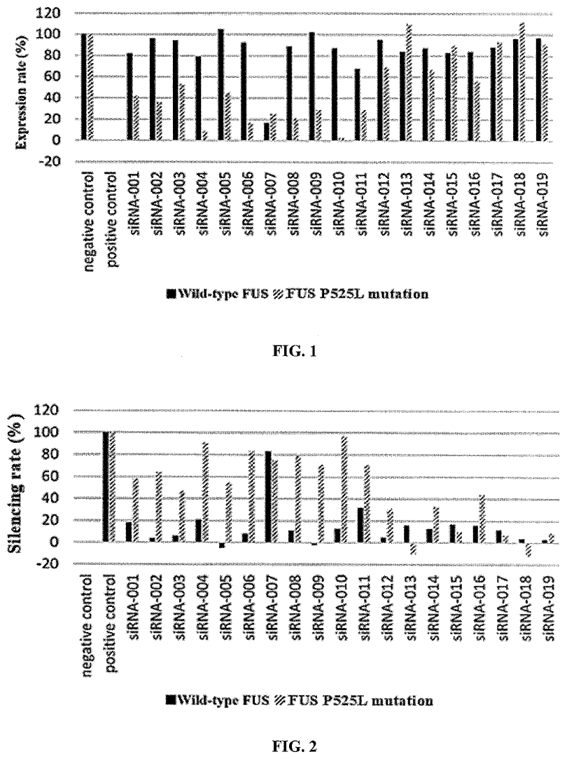

The expression rate of wild-type FUS/FUS P525L mutation and selectivity of the 21 mer siRNA shown in FIG. 1 are shown in Table 2.

TABLE 2

| | The expression | The expression | Selectivity (1) |
|---|---|---|---|
| | rate of | rate of FUS | Wild-type FUS/FUS |
| | wild-type FUS | P525L mutation | P525L mutation |
| siRNA-001 | 82 | 42 | 2.0 |
| siRNA-002 | 96 | 36 | 2.7 |
| siRNA-003 | 94 | 53 | 1.8 |
| siRNA-004 | 79 | 9 | 8.8 |
| siRNA-005 | 105 | 45 | 2.3 |
| siRNA-006 | 92 | 17 | 5.4 |
| siRNA-007 | 17 | 25 | 0.7 |
| siRNA-008 | 89 | 21 | 4.2 |
| siRNA-009 | 102 | 29 | 3.5 |
| siRNA-010 | 87 | 3 | 29.0 |
| siRNA-011 | 68 | 29 | 2.3 |
| siRNA-012 | 95 | 69 | 1.4 |
| siRNA-013 | 84 | 110 | 0.8 |
| siRNA-014 | 87 | 67 | 1.3 |
| siRNA-015 | 83 | 90 | 0.9 |
| siRNA-016 | 84 | 56 | 1.5 |
| siRNA-017 | 88 | 93 | 0.9 |
| siRNA-018 | 96 | 112 | 0.9 |
| siRNA-019 | 97 | 91 | 1.1 |

The silencing rate of wild-type FUS, the silencing rate of FUS P525L mutation, and selectivity of the 21 mer siRNA shown in FIG. 2 are shown in Table 3.

TABLE 3

| | | | Selectivity (2) |
|---|---|---|---|
| | The silencing rate of | The silencing rate of | Wild-type FUS -FUS P525L |
| | wild-type FUS | FUS P525L mutation | mutation |
| siRNA-001 | 18 | 58 | 40 |
| siRNA-002 | 4 | 64 | 60 |
| siRNA-003 | 6 | 47 | 41 |
| siRNA-004 | 21 | 91 | 70 |
| siRNA-005 | −5 | 55 | 60 |
| siRNA-006 | 8 | 83 | 75 |
| siRNA-007 | 83 | 75 | −8 |
| siRNA-008 | 11 | 79 | 68 |
| siRNA-009 | −2 | 71 | 73 |
| siRNA-010 | 13 | 97 | 84 |
| siRNA-011 | 32 | 71 | 39 |
| siRNA-012 | 5 | 31 | 26 |
| siRNA-013 | 16 | −10 | −26 |
| siRNA-014 | 13 | 33 | 20 |
| siRNA-015 | 17 | 10 | −7 |
| siRNA-016 | 16 | 44 | 28 |
| siRNA-017 | 12 | 7 | −5 |
| siRNA-018 | 4 | −12 | −16 |
| siRNA-019 | 3 | 9 | 6 |

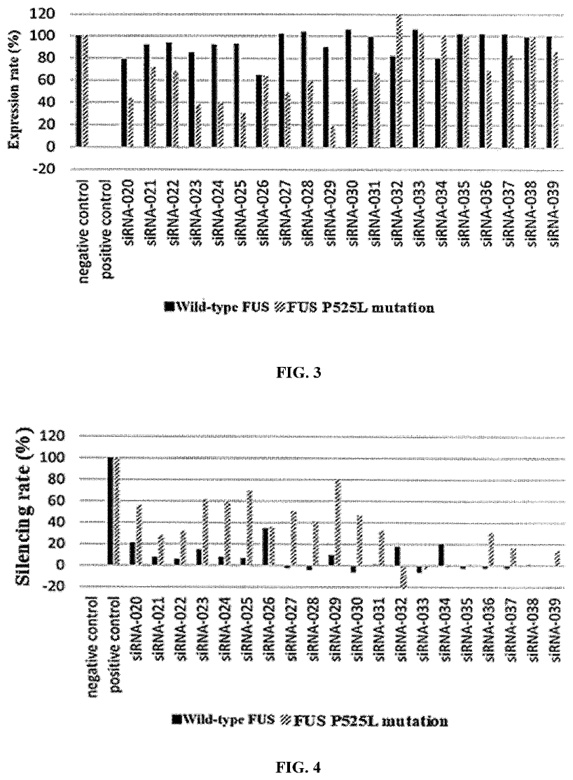

The wild-type FUS/FUS P525L mutation expression rates and selectivity of the 22 mer siRNA shown in FIG. 3 are shown in Table 4.

TABLE 4

| | | The expression | Selectivity (1) |
|---|---|---|---|
| | The expression | rate of | Wild-type |
| | rate of | FUS P525L | FUS/FUS |
| | wild-type FUS | mutation | P525L mutation |
| siRNA-020 | 79 | 44 | 1.8 |
| siRNA-021 | 92 | 72 | 1.3 |
| siRNA-022 | 94 | 68 | 1.4 |
| siRNA-023 | 85 | 38 | 2.2 |
| siRNA-024 | 92 | 40 | 2.3 |
| siRNA-025 | 93 | 30 | 3.1 |
| siRNA-026 | 65 | 64 | 1.0 |
| siRNA-027 | 102 | 49 | 2.1 |
| siRNA-028 | 104 | 59 | 1.8 |
| siRNA-029 | 90 | 19 | 4.7 |
| siRNA-030 | 106 | 53 | 2.0 |
| siRNA-031 | 99 | 67 | 1.5 |
| siRNA-032 | 82 | 122 | 0.7 |
| siRNA-033 | 106 | 103 | 1.0 |
| siRNA-034 | 80 | 101 | 0.8 |
| siRNA-035 | 102 | 99 | 1.0 |
| siRNA-036 | 102 | 69 | 1.5 |
| siRNA-037 | 102 | 83 | 1.2 |
| siRNA-038 | 99 | 99 | 1.0 |
| siRNA-039 | 100 | 86 | 1.2 |

The silencing rate of wild-type FUS, the silencing rate of FUS P525L mutation, and selectivity of the 22 mer siRNA shown in FIG. 4 are shown in Table 5.

TABLE 5

| | | The silencing | Selectivity (2) |
|---|---|---|---|
| | The silencing | rate of | Wild-type |
| | rate of | FUS P525L | FUS - FUS |
| | wild-type FUS | mutation | P525L mutation |
| siRNA-020 | 21 | 56 | 35 |
| siRNA-021 | 8 | 28 | 20 |
| siRNA-022 | 6 | 32 | 26 |
| siRNA-023 | 15 | 62 | 47 |
| siRNA-024 | 8 | 60 | 52 |
| siRNA-025 | 7 | 70 | 63 |
| siRNA-026 | 35 | 36 | 1 |
| siRNA-027 | −2 | 51 | 53 |
| siRNA-028 | −4 | 41 | 45 |
| siRNA-029 | 10 | 81 | 71 |
| siRNA-030 | −6 | 47 | 53 |
| siRNA-031 | 1 | 33 | 32 |
| siRNA-032 | 18 | −22 | −40 |
| siRNA-033 | −6 | −3 | 3 |
| siRNA-034 | 20 | −1 | −21 |
| siRNA-035 | −2 | 1 | 3 |
| siRNA-036 | −2 | 31 | 33 |
| siRNA-037 | −2 | 17 | 19 |
| siRNA-038 | 1 | 1 | 0 |
| siRNA-039 | 0 | 14 | 14 |

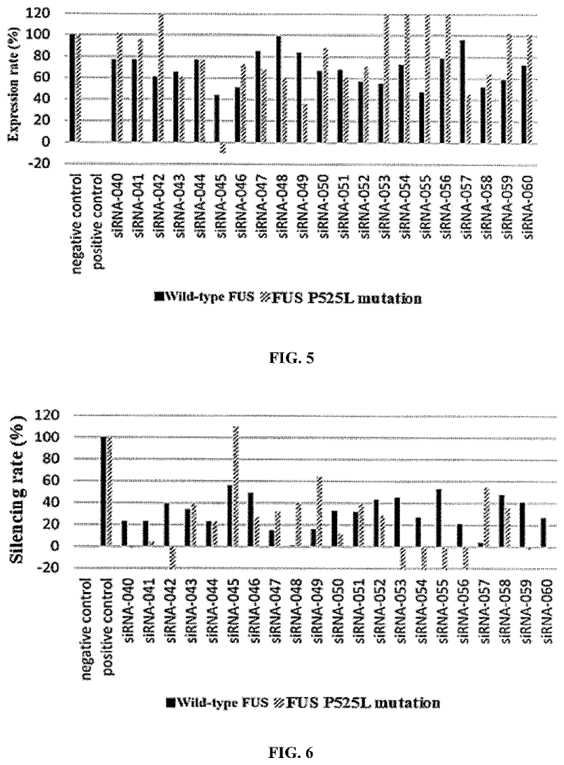

The wild-type FUS/FUS P525L mutation expression rates and selectivity of the 23 mer siRNA shown in FIG. 5 are shown in Table 6.

TABLE 6

| | | The expression | Selectivity (1) |
|---|---|---|---|
| | The expression | rate of | Wild-type |
| | rate of | FUS P525L | FUS/FUS |
| | wild-type FUS | mutation | P525L mutation |
| siRNA-040 | 77 | 101 | 0.8 |
| siRNA-041 | 77 | 96 | 0.8 |
| siRNA-042 | 61 | 138 | 0.4 |
| siRNA-043 | 66 | 61 | 1.1 |
| siRNA-044 | 77 | 77 | 1.0 |
| siRNA-045 | 44 | −10 | >40.0 |
| siRNA-046 | 51 | 73 | 0.7 |
| siRNA-047 | 85 | 68 | 1.3 |
| siRNA-048 | 99 | 60 | 1.7 |
| siRNA-049 | 84 | 36 | 2.3 |
| siRNA-050 | 67 | 88 | 0.8 |
| siRNA-051 | 68 | 61 | 1.1 |
| siRNA-052 | 57 | 71 | 0.8 |
| siRNA-053 | 55 | 137 | 0.4 |
| siRNA-054 | 73 | 198 | 0.4 |
| siRNA-055 | 47 | 123 | 0.4 |
| siRNA-056 | 79 | 134 | 0.6 |
| siRNA-057 | 96 | 45 | 2.1 |
| siRNA-058 | 52 | 64 | 0.8 |
| siRNA-059 | 59 | 102 | 0.6 |
| siRNA-060 | 73 | 101 | 0.7 |

The silencing rate of wild-type FUS, the silencing rate of FUS P525L mutation, and selectivity of the 23 mer siRNA shown in FIG. 6 are shown in Table 7.

TABLE 7

| | | The silencing | Selectivity (2) |
|---|---|---|---|
| | The silencing | rate of | Wild-type |
| | rate of | FUS P525L | FUS - FUS |
| | wild-type FUS | mutation | P525L mutation |
| siRNA-040 | 23 | −1 | −24 |
| siRNA-041 | 23 | 4 | −19 |
| siRNA-042 | 39 | −38 | −77 |
| siRNA-043 | 34 | 39 | 5 |
| siRNA-044 | 23 | 23 | 0 |
| siRNA-045 | 56 | 110 | 54 |
| siRNA-046 | 49 | 27 | −22 |
| siRNA-047 | 15 | 32 | 17 |
| siRNA-048 | 1 | 40 | 39 |
| siRNA-049 | 16 | 64 | 48 |
| siRNA-050 | 33 | 12 | −21 |
| siRNA-051 | 32 | 39 | 7 |
| siRNA-052 | 43 | 29 | −14 |
| siRNA-053 | 45 | −37 | −82 |
| siRNA-054 | 27 | −98 | −125 |
| siRNA-055 | 53 | −23 | −76 |
| siRNA-056 | 21 | −34 | −56 |
| siRNA-057 | 4 | 55 | 51 |
| siRNA-058 | 48 | 36 | −12 |
| siRNA-059 | 41 | −2 | −43 |
| siRNA-060 | 27 | −1 | −28 |

The siRNA of the present invention exhibits silencing of FUS P525L mutation and, above all, it selectively exhibits silencing of FUS P525L mutation without substantially silencing of wild-type FUS. Here, “without substantially silencing of wild-type FUS” means that undesirable symptoms due to silencing of wild-type FUS do not substantially appear and that it selectively exhibits silencing of FUS P525L mutation. Specifically, it is defined as having such selectivity that the expression rate of wild-type FUS is usually 1.5 times or more, preferably 2 times or more than the expression rates of FUS P525L mutation as shown in Tables 2, 4, and 6 above and that the silencing rate of FUS P525L mutation is usually 20% or more, preferably 40% or more than the silencing rates of wild-type FUS as shown in Tables 3, 5, and 7 above. The selectivity can be evaluated separately or in combination.

As used in the present invention, the siRNA which selectively exhibits silencing of FUS P525L mutation, for example, is the siRNA comprising a sequence identical or substantially identical to SEQ ID NO: 2, SEQ ID NO: 4, SEQ ID NO: 6, SEQ ID NO: 8, SEQ ID NO: 10, SEQ ID NO: 12, SEQ ID NO: 16, SEQ ID NO: 18, SEQ ID NO: 20, SEQ ID NO: 22, SEQ ID NO: 24, SEQ ID NO: 28, or SEQ ID NO: 32 as an antisense strand, preferably, the siRNA comprising a sequence identical or substantially identical to SEQ ID NO: 2, SEQ ID NO: 4, SEQ ID NO: 6, SEQ ID NO: 8, SEQ ID NO: 10, SEQ ID NO: 12, SEQ ID NO: 16, SEQ ID NO: 18, SEQ ID NO: 20, SEQ ID NO: 22, or SEQ ID NO: 32 as an antisense strand.

In addition, the examples of the siRNA used in the present invention include the siRNA comprising a sequence identical or substantially identical to SEQ ID NO: 1/SEQ ID NO: 2, SEQ ID NO: 3/SEQ ID NO: 4, SEQ ID NO: 5/SEQ ID NO: 6, SEQ ID NO: 7/SEQ ID NO: 8, SEQ ID NO: 9/SEQ ID NO: 10, SEQ ID NO: 11/SEQ ID NO: 12, SEQ ID NO: 15/SEQ ID NO: 16, SEQ ID NO: 17/SEQ ID NO: 18, SEQ ID NO: 19/SEQ ID NO: 20, SEQ ID NO: 21/SEQ ID NO: 22, SEQ ID NO: 23/SEQ ID NO: 24, SEQ ID NO: 27/SEQ ID NO: 28, SEQ ID NO: 31/SEQ ID NO: 32, SEQ ID NO: 39/SEQ ID NO: 40, SEQ ID NO: 41/SEQ ID NO: 42, SEQ ID NO: 43/SEQ ID NO: 44, SEQ ID NO: 45/SEQ ID NO: 46, SEQ ID NO: 47/SEQ ID NO: 48, SEQ ID NO: 49/SEQ ID NO: 50, SEQ ID NO: 53/SEQ ID NO: 54, SEQ ID NO: 55/SEQ ID NO: 56, SEQ ID NO: 57/SEQ ID NO: 58, SEQ ID NO: 59/SEQ ID NO: 60, SEQ ID NO: 61/SEQ ID NO: 62, SEQ ID NO: 71/SEQ ID NO: 72, SEQ ID NO: 89/SEQ ID NO: 90, SEQ ID NO: 95/SEQ ID NO: 96, SEQ ID NO: 97/SEQ ID NO: 98, SEQ ID NO: 113/SEQ ID NO: 114 as a sense strand/an antisense strand, more preferably, the siRNA comprising a sequence identical or substantially identical to SEQ ID NO: 1/SEQ ID NO: 2, SEQ ID NO: 3/SEQ ID NO: 4, SEQ ID NO: 5/SEQ ID NO: 6, SEQ ID NO: 7/SEQ ID NO: 8, SEQ ID NO: 9/SEQ ID NO: 10, SEQ ID NO: 11/SEQ ID NO: 12, SEQ ID NO: 15/SEQ ID NO: 16, SEQ ID NO: 17/SEQ ID NO: 18, SEQ ID NO: 19/SEQ ID NO: 20, SEQ ID NO: 21/SEQ ID NO: 22, SEQ ID NO: 45/SEQ ID NO: 46, SEQ ID NO: 47/SEQ ID NO: 48, SEQ ID NO: 49/SEQ ID NO: 50, SEQ ID NO: 53/SEQ ID NO: 54, SEQ ID NO: 55/SEQ ID NO: 56, SEQ ID NO: 57/SEQ ID NO: 58, SEQ ID NO: 59/SEQ ID NO: 60, SEQ ID NO: 89/SEQ ID NO: 90, SEQ ID NO: 97/SEQ ID NO: 98, SEQ ID NO: 113/SEQ ID NO: 114 as a sense strand/an antisense strand.

As used herein, a substantially identical sequence has an overhang of at least one or more nucleotides (preferably 2 nucleotides) at the 3′ ends of a sense strand or/and an antisense strand.

The siRNA of the present invention is useful as an ALS therapeutic agent, and its therapeutic effect can be evaluated using methods described in the following documents or methods equivalent thereto.
1. J Clin Invest, McCampbell A. et al. p 3558-3567: 2018
2. E Bio Medicine, Akiyama T. et al. p 362-378: 2019
3. BRAIN, Shiihashi G. et al. p 2380-2394: 2016

The pharmaceutical compositions of the present invention may be used as they are for treatment of ALS or may be formulated into various dosage forms in a manner known to those skilled in the art using a pharmaceutically acceptable carrier or excipient. The carriers or excipients used are known to those skilled in the art and may be selected as appropriate. The agents of the present invention can be produced using the means and methods known to those skilled in the art.

The ALS therapeutic agents of the present invention can be formulated by mixing, dissolving, granulating, tableting, emulsifying, encapsulating, lyophilizing the siRNA targeting the FUS P525L mutation with a pharmaceutically acceptable carrier well known in the art.

For oral administration, the siRNA targeting the FUS P525L mutation can be formulated together with a pharmaceutically acceptable solvent, excipient, binder, stabilizer, dispersant, etc. into dosage forms such as tablets, pills, sugar coated tablets, soft capsules, hard capsules, solutions, suspensions, emulsions, gels, syrups, slurries, etc.

For parenteral administration, the siRNA targeting the FUS P525L mutation can be formulated together with a pharmaceutically acceptable solvent, excipient, binder, stabilizer, dispersant, etc. into dosage forms, such as injectable solutions, suspensions, emulsions, creams, ointments, inhalants, suppositories, etc.

For an injectable formulation, the siRNA targeting the FUS P525L mutation can be dissolved in an aqueous solution, preferably in a physiologically compatible buffer, such as Hank's solution, Ringer's solution, or physiological saline buffer. In addition, the composition may be in the form of suspension, solution, or emulsion in an oily or aqueous vehicle. Alternatively, a therapeutic agent may be produced in the form of a powder and an aqueous solution or suspension may be prepared using sterile water or the like prior to use. For administrations by inhalation, the siRNA targeting the FUS P525L mutation can be powdered together with a suitable base such as lactose or starch into a powder mixture.

Furthermore, the siRNA targeting the FUS P525L mutation can be administered in the form of a non-viral vector or viral vector. Such dosage forms may be produced using known methods such as those described in Separate Volume of Laboratory Medicine, “Basic Technology of Gene Therapy”, Yodosha, 1996; Separate Volume of Laboratory Medicine, “Gene Transfer & Experimental Methods of Expression Analysis”, Yodosha, 1997.

The dosage and number of doses vary depending on dosage form and route of administration, as well as the symptoms, age, and weight of a patient. However, in general, the siRNA targeting the FUS P525L mutation can be administered from once to several times a day, in a range from about 0.001 mg to 1000 mg, preferably from about 0.01 mg to 10 mg per day and 1 kg of body weight.

In yet another embodiment, the present invention provides the use of the above compositions for producing the ALS therapeutic agents.

In yet another embodiment, the present invention provides the use of the above compositions for treating ALS.

In yet another embodiment, the present invention provides a method of treatment comprising administering the above compositions to patients with ALS.

The ALS is preferably juvenile ALS with P525L mutation in FUS gene.

In another embodiment, the present invention provides a DNA vector for expressing the siRNA of the present invention in cells, a pharmaceutical composition comprising the DNA vector and a pharmaceutically acceptable carrier, and the cells comprising the DNA vector.

In another embodiment, the present invention provides a method for screening the ALS therapeutic agents that selectively silence FUS P525L mutation, characterized by determining the silencing rate of wild-type FUS and the silencing rate of FUS P525L mutation.

Examples of the ALS therapeutic agents include nucleic acid molecules, peptides, proteins, non-peptidic compounds, synthetic compounds, fermentation products, cell extracts, extracts of a plant, extracts of an animal tissue, plasma, and the like. These compounds may be novel or known compounds.

In another embodiment, the present invention provides a method for screening the siRNA for use as an active ingredient of the ALS therapeutic agents. The method comprises
(1) a step of designing the siRNAs comprising a region complementary or substantially complementary to a portion of mRNA encoding FUS P525L mutation, wherein the complementary region is 19 to 21 nucleotides in length,
(2) a step of producing the siRNAs designed in step (1), and
(3) a step of screening for the siRNA that selectively exhibits silencing of FUS P525L mutation without substantially silencing of wild-type FUS, from the siRNAs produced in step (2).

In another embodiment, the present invention provides a method for producing an ALS therapeutic agent comprising, as an active ingredient, the siRNA that selectively exhibits silencing of FUS P525L mutation. The method comprises
(1) a step of designing the siRNAs comprising a region complementary or substantially complementary to a portion of mRNA encoding FUS P525L mutation, wherein the complementary region is 19 to 21 nucleotides in length,
(2) a step of producing the siRNAs designed in step (1),
(3) a step of screening for the siRNA that selectively exhibits silencing of FUS P525L mutation without substantially silencing of wild-type FUS, from the siRNAs produced in step (2),
(4) a step of producing a pharmaceutical composition containing the siRNA screened in step (3) as an active ingredient, and
(5) a step of confirming the effect of the pharmaceutical composition produced in step (4) as the ALS therapeutic agent.

The present invention will now be described by way of examples. However, the invention is not limited to the following examples.

### Example 1

Synthesis of human FUS_{wild-type} and FUS_{P525L} cDNA The cDNA sequence of human FUS P525L mutation (as FUS_{P525L} hereinafter) is shown in Sequence 1 and the cDNA sequence of human wild-type FUS (as FUS_{wild-type} hereinafter) is shown in Sequence 2.

Artificial genes are provided by Genscript Japan Co. Ltd.

It should be noted that the synthetic genes were inserted into BamHI/XhoI sites in the multi-cloning site of the pcDNA3.1+ vector.

Sequence 1
(SEQ ID NO: 246)
tgcgcggacatggcctcaaacgattatacccaacaagcaacccaa
agctatggggcctaccccacccagcccgggcagggctattcccag
cagagcagtcagccctacggacagcagagttacagtggttatagc
cagtccacggacacttcaggctatggccagagcagctattcttct
tatggccagagccagaacagctatggaactcagtcaactccccag
ggatatggctcgactggggctatggcagtagccagagctcccaat
cgtcttacgggcagcagtcctcctatcctggctatggccagcagc
cagctcccagcagcacctcgggaagttacggtagcagttctcaga
gcagcagctatgggcagccccagagtgggagctacagccagcagc
ctagctatggtggacagcagcaaagctatggacagcagcaaagct
ataatccccctcagggctatggacagcagaaccagtacaacagca
gcagtggtggtggaggtggaggtggaggtggaggtaactatggcc
aagatcaatcctccatgagtagtggtggtggcagtggtggcggtt
atggcaatcaagaccagagtggtggaggtggcagcggtggctatg
gacagcaggaccgtggaggccgcggcaggggtggcagtggtggcg
gcggcggcggcggcggtggtggttacaaccgcagcagtggtggct
atgaacccagaggtcgtggaggtggccgtggaggcagaggtggca
tgggcggaagtgaccgtggtggcttcaataaatttggtggccctc
gggaccaaggatcacgtcatgactccgaacaggataattcagaca
acaacaccatctttgtgcaaggcctgggtgagaatgttacaattg
agtctgtggctgattacttcaagcagattggtattattaagacaa
acaagaaaacgggacagcccatgattaatttgtacacagacaggg
aaactggcaagctgaagggagaggcaacggtctcttttgatgacc
caccttcagctaaagcagctattgactggtttgatggtaaagaat
tctccggaaatcctatcaaggtctcatttgctactcgccgggcag
actttaatcggggggggcaatggtcgtggaggccgagggcgagga
ggacccatgggccgtggaggctatggaggtggtggcagtggtggt
ggtggccgaggaggatttcccagtggaggtggtggcggtggagga
cagcagcgagctggtgactggaagtgtcctaatcccacctgtgag
aatatgaacttctcttggaggaatgaatgcaaccagtgtaaggcc
cctaaaccagatggcccaggagggggaccaggtggctctcacatg
gggggtaactacggggatgatcgtcgtggtggcagaggaggctat
gatcgaggcggctaccggggccgcggggggaccgtggaggcttcc
gagggggccggggtggtggggacagaggtggctttggccctggca
agatggattccaggggtgagcacagacaggatcgcagggagaggc
cgtattaattagcctggctccccaggttctggaacagctttttgt
cctgtacccagtgttaccctcgttattttgtaaccttccaattcc
tgatcacccaagggtttttttgtgtcggactatgtaattgtaact
atacctctggttcccattaaaagtgaccattttagttaaaaaaaa
Sequence 2
(SEQ ID NO: 247)
tgcgcggacatggcctcaaacgattatacccaacaagcaacccaa
agctatggggcctaccccacccagcccgggcagggctattcccag
cagagcagtcagccctacggacagcagagttacagtggttatagc
cagtccacggacacttcaggctatggccagagcagctattcttct
tatggccagagccagaacagctatggaactcagtcaactccccag
ggatatggctcgactggcggctatggcagtagccagagctcccaa
tcgtcttacgggcagcagtcctcctatcctggctatggccagcag
ccagctcccagcagcacctcgggaagttacggtagcagttctcag
agcagcagctatgggcagccccagagtgggagctacagccagcag
cctagctatggtggacagcagcaaagctatggacagcagcaaagc
tataatccccctcagggctatggacagcagaaccagtacaacagc
agcagtggtggtggaggtggaggtggaggtggaggtaactatggc
caagatcaatcctccatgagtagtggtggtggcagtggtggcggt
tatggcaatcaagaccagagtggtggaggtggcagcggtggctat
ggacagcaggaccgtggaggccgcggcaggggtggcagtggtggc
ggcggcggcggcggcggtggtggttacaaccgcagcagtggtggc
tatgaacccagaggtcgtggaggtggccgtggaggcagaggtggc
atgggcggaagtgaccgtggtggcttcaataaatttggtggccct
cgggaccaaggatcacgtcatgactccgaacaggataattcagac
aacaacaccatctttgtgcaaggcctgggtgagaatgttacaatt
gagtctgtggctgattacttcaagcagattggtattattaagaca
aacaagaaaacgggacagcccatgattaatttgtacacagacagg
gaaactggcaagctgaagggagaggcaacggtctcttttgatgac
ccaccttcagctaaagcagctattgactggtttgatggtaaagaa
ttctccggaaatcctatcaaggtctcatttgctactcgccgggca
gactttaatcggggtggtggcaatggtcgtggaggccgagggcga
ggaggacccatgggccgtggaggctatggaggtggtggcagtggt
ggtggtggccgaggaggatttcccagtggaggtggtggcggtgga
ggacagcagcgagctggtgactggaagtgtcctaatcccacctgt
gagaatatgaacttctcttggaggaatgaatgcaaccagtgtaag
gcccctaaaccagatggcccaggagggggaccaggtggctctcac
atggggggtaactacggggatgatcgtcgtggtggcagaggaggc
tatgatcgaggcggctaccggggccgcggcggggaccgtggaggc
ttccgagggggccggggtggtggggacagaggtggctttggccct
ggcaagatggattccaggggtgagcacagacaggatcgcagggag
aggctgtattaattagcctggctccccaggttctggaacagcttt
ttgtcctgtacccagtgttaccctcgttattttgtaaccttccaa
ttcctgatcacccaagggtttttttgtgtcggactatgtaattgt
aactatacctctggttcccattaaaagtgaccattttagttaaaa
aaaa

### Example 2

### Construction of GFP-Fused FUS_{wild-type} and FP635-Fused FUS_{P525L} Gene Expression Vectors

PCR was carried out using the primer sets shown in Table 8 for pTurboGFP vector, pTurboFP635 vector, and the artificial genes prepared in Example 1 “Synthesis of human FUS_{wild-type} and FUS_{P525L} cDNA”.

25 μl of PrimSTAR Max Premix (Takara Bio Inc.), 4 μL of 2.5 μM Primer (final concentration of 0.2 μM), 1 μL of template (20 ng), and 20 μL of water were mixed and incubated at 98° C. for 10 seconds. Then, the PCR was carried out for 35 cycles at 98° C. for 10 seconds, at 55° C. for 5 seconds, and at 72° C. for 10 seconds (25 seconds when the vector was used as template).

The vector-amplified fragment and the FUS gene-amplified fragment were linked by In-Fusion reaction. In other words, 2 μL of the inserted fragment, 1 μL of the vector amplicon, 2 μL of 5× In-Fusion HD Enzyme Premix (Takara Bio Inc.), and 5 μL of water were mixed, allowed to react at 50° C. for 15 minutes, and transformed into NEB Turbo Competent E. coli (New England Biolabs Japan). Plasmid vectors were prepared from the transformants, and it was confirmed from their DNA sequences that the desired cDNA had been properly inserted.

The FUS_{wild-type} was cloned with Turbo GFP fluorescent protein at its N-terminus in-frame with pTurboGFP vector (Evrogen), and the FUS_{P525L} was cloned with Turbo FP635 fluorescent protein at its N-terminus in flame with pTurboFP635 vector (Evrogen).

TABLE 8

| | | SEQ |
|---|---|---|
| Name of | | ID |
| template | Sequence of primer | NO |
| pTurboGFP- | Forward Primer: | 248 |
| c vector | TCTCGAGCTCAAGCTT | |
| | CGAATTCTG | |
| | Reverse Primer: | |
| | TCTTTCTTCACCGGC | 249 |
| | ATCTGCATCC | |
| pcDNA3.1(+)_ | Forward Primer: | 250 |
| FUS_{Wild-type} | GCCGGTGAAGAAAGAATG | |
| | GCCTCAAACGATTATAC | |
| | Reverse Primer: | 251 |
| | AGCTTGAGCTCGAGATTTT | |
| | TTTTAACTAAAATGGTCAC | |
| pTurboFP635- | Forward Primer: | 252 |
| c vector | TCTCGAGCTCAAGCTTCGA | |
| | ATTCTG | |
| | Reverse Primer: | 253 |
| | TCTGAGTCCGGAGCTGTGC | |
| | CCCAGT | |
| pcDNA3.1(+)_ | Forward | 254 |
| FUS_{P525L} | Primer: | |
| | AGCTCCGGACTCAGAATG | |
| | GCCTCAAACGATTATAC | |
| | Reverse | 255 |
| | Primer: AGCTTGAGCTCGAGATTTT | |
| | TTTTAACTAAAATGGTCAC | |

### Example 3

Production of TurboGFP-fused FUS_{wild type} and TurboFP635-fused FUS_{P525L} stable expression HEK293 cell lines

TurboGFP-fused FUS_{wild-type} cDNA or TurboFP635-fused FUS_{P525L} cDNA was inserted into the AAVS1 region, which is a safe harbor on the HEK293 cell genome, to produce stable expression cell lines.

In the production of the stable expressing cell lines, the AAS1 transgene knockin vector kit (Origene) was used.

PCR was carried out using the templates and primer sets shown in Table 9 for sequences containing each FUS cDNA from the initiation codon of each fluorescent protein of the previously prepared TurboGFP-fused FUS_{wild-type} expression vector and TurboFP635-fused FUS_{P525L} expression vector.

25 μL of PrimSTAR Max Premix (2×), 4 μL each of 2.5 μM Primer (final concentration of 0.2 μM), 1 μL of template (20 ng), and 20 μL of water were mixed and incubated at 98° C. for 10 seconds. Then, the PCR was carried out for 35 cycles at 98° C. for 10 seconds, at 55° C. for 5 seconds, and at 72° C. for 10 seconds. Each PCR product was purified using a GFX® PCR DNA and Gel Band Purification Kit (Global Life Science Technologies Japan Co. Ltd.).

The purified PCR products and pAAVS1-puro-DNR plasmid vectors (Origene) were digested with restriction enzymes Asci and NotI-HF (both from New England Biolabs Japan) and then purified using a GFX© PCR DNA and Gel Band Purification Kit. A ligation reaction (Ligation high ver. 2, Toyobo Co., Ltd.) was performed between the plasmid vector and the inserted cDNA, and transformed into NEB Turbo Competent E. coli. Plasmid vectors were prepared from the transformants and it was confirmed from their DNA sequences that the desired cDNA had been properly inserted.

The prepared pAAVS1-puro-DNR plasmid vector was co-transfected with the pCAS-Guide-AAS1 plasmid vector by electroporation (4D-Nucleofector system, ADI 4D-Nucleofector® Y kit, program code CA-215, Lonza). After drug selection with puromycin (3 g/mL) for about 1 week from 48 hours after the transfection, cloning was performed using the luminescence of TurboGFP or TurboFP635.

TABLE 9

| Name of | Sequence of | SEQ ID |
|---|---|---|
| template | primer | NO |
| pTurboGFP_ | Forward | 256 |
| FUS_{Wild-type} | Primer: | |
| | ATTAGGCGCGCCACC | |
| | ATGGAGAGCGACGAG | |
| | Reverse Primer: | 257 |
| | TAATGCGGCCGCTTG | |
| | AGCTCGAG | |
| pTurboFP635_ | Forward Primer: | 258 |
| FUS_{P525L} | ATTAGGCGCGCCACC | |
| | ATGGTGGGTGAGGAT | |
| | AG | |
| | Reverse Primer: | 259 |
| | TAATGCGGCCGCTTG | |
| | AGCTCGAG | |

### Example 4

### Cell Culture

HEK293 cells were cultured at 37° C. and 5% CO₂ using Advanced DMEM (Thermo Fisher Scientific Co. Ltd.) containing 10% FBS, 4 mM GlutaMAX© Supplement.

The HEK293 cells used (cell number: JCRB9068) were purchased from the JCRB Cell Bank in the Culture Resource Laboratory of the Institute of Biomedical Innovation, Health and Nutrition, a national research and development agency.

### Example 5

### Preparation of Evaluation Samples of RNA Interference Using the Stable Expression Strains

siRNA001 to siRNA060 comprised of the sense strands/antisense strands of SEQ ID NOs: 121 to 240 shown in Table 1. were produced according to methods well known in the field of nucleic acid synthesis (consignment to GeneDesign, Inc. for the production).

The negative control siRNA is the siRNA wherein the sequence is not similar with known sequences of human, mouse, or rat's genes, and is provided by Horizon Discovery. Its sequence is UAGCGACUAAACACAUCAA (SEQ ID NO: 241). On the other hand, the positive control siRNA is a mixture of four kinds of siRNAs (SEQ ID NOS: 242 to 245) designed to target any region of human FUS mRNA and is provided by Horizon Discovery. The sequences of the four kinds are CCUACGGACAGCAGUUA (SEQ ID NO: 242), GAUUAUACCCAACAGCAA (SEQ ID NO: 243), GAUCAUCCCAUCAGUA (SEQ ID NO: 244), and CGGGACAGCCAGAUUAA (SEQ ID NO: 245).

0.12 μL each of various siRNAs (siRNA001 to siRNA060, 10 μM) were added to 20 μL of Opti-MEM© I Reduced-Serum Medium and stirred gently. To each of these solutions, 0.2 μL of Lipofectamine® RNAiMAX Transfection Reagent was added and mixed gently, and then incubated at room temperature for 10 to 20 minutes.

TurboGFP-fused FUS_{wild-type} stable expressing HEK293 cell line and TurboFP635-fused FUS_{P525L} stable expressed HEK293 cell line were mixed at 1:1 and diluted with FluoroBrite® DMEM containing 10% FBS, 4 mM GlutaMax® Supplement so that the concentration was 5-8×10⁴ cells/mL.

To Cell Carrier-96ultra (Black, 96 wells, clear bottom, with lid, collagen coated, PerkinElmer), 100 μL of the diluted cells and 20.32 μL of the complex of siRNA/Lipofectamine® RNAiMAX were added, mixed and cultured at 37° C. in a 5% CO₂ incubator for 48 hours (the final concentration of siRNA was 1 nM/well). About 48 hours after the transfection, NucBlue® Live ReadyProbes® Reagent (Thermo Fisher Scientific) was added at 2 μL/well and stirred gently, and then incubated at room temperature for 30 minutes to stain the nucleus.

The cells were added with 100 μL of 10% neutrally buffered formalin solution (Sigma-Aldrich) and stirred gently, and then immobilized by incubation at room temperature for 1 hour.

### Example 6

Analysis of Expression Amount of FUS_{wild-type} and FUS_{P525L}

Images were acquired with an Operetta CLS® High Content Confocal Imaging System (PerkinElmer) equipped with a 20-fold water immersion lens at confocal mode using Hoechst33342 (maximum excitation wavelength: 461 nm) as a reporter protein of the nucleus, TurboGFP (maximum excitation wavelength: 452 nm) as a reporter protein of wild-type FUS and TurboFP635 (maximum excitation wavelength: 588 nm) as a reporter protein of mutant FUS. Furthermore, the fluorescence intensity of TurboGFP in the nuclear region and the fluorescence intensity of TurboFP635 in the cytoplasmic region were determined respectively by using the image analysis software Harmony© (Ver. 4.9, PerkinElmer), and the TurboGFP-positive cells (fluorescence intensity of 1500 or higher) and TurboFP635-positive cells (fluorescence intensity of 800 or higher) were selected. TurboGFP-positive cell rates (TurboGFP-positive cell count/total cell count) and TurboFP635-positive cell rates (TurboFP635-positive cell count/total cell count) were calculated from the selected cells. Then, the relative values of the expression rate and the silencing rate were calculated from the values of the control as follows.

$$( Expression Rate ) = 100 \times \{ ( Turbo GFP - positive cell rate after treatment of various siRNAs ( siRNA 001 to siRNA 060 ) ) - ( Turbo GFP - positive cell rate after trearment of positive control siRNA ) \} \text{} / \{ ( Turbo GFP - positive cell rate after treatment of negative control siRNA ) - ( Turbo GFP - positive cell rate after treatment of positive control siRNA ) \}$$

$$( Silencing Rate ) = 100 \times \{ ( Turbo GFP - positive - cell rate after treatment of various siRNAs ( siRNA 001 to siRNA 060 ) ) - ( Turbo GFP - positive cell rate after trearment of negative control siRNA ) \} \text{} / \{ ( Turbo GFP - positive cell rate after treatment of positive control siRNA ) - ( Turbo GFP - positive cell rate after treatment of negative control siRNA ) \}$$

The expression rates are shown in FIGS. 1, 3, and 5. The silencing rates are shown in FIGS. 2, 4, and 6.

It was conformed from the results of FIGS. 1 to 6, that siRNA-001, 002, 003, 004, 005, 006, 008, 009, 010, 011, 012, 014, 016, 020, 021, 022, 023, 024, 025, 027, 028, 029, 030, 031, 036, 045, 048, 049, and 057 are the siRNAs that selectively exhibits silencing of FUS P525L mutation over wild-type FUS.

Therefore, it is particularly expected that the siRNA or the like composed of an antisense strand comprising a sequence identical or substantially identical to SEQ ID NO: 2, SEQ ID NO: 4, SEQ ID NO: 6, SEQ ID NO: 8, SEQ ID NO: 10, SEQ ID NO: 12, SEQ ID NO: 16, SEQ ID NO: 18, SEQ ID NO: 20, SEQ ID NO: 22, SEQ ID NO: 24, SEQ ID NO: 28, or SEQ ID NO: 32 excluding the overhang (dTdT) from the antisense strand of the above siRNA, as the siRNA of the present invention, will be useful as therapeutic agents for the ALS associated with the FUS P525L mutation gene.

### Example 7

### Production of FUS Knockout (KO) HEK293 Cell Lines

FUS/TLS CRISPR/CAS9KO (sc-400612) plasmids (Santa Cruz) and FUS/TLS HDR plasmids (h) (sc-400612-HDR) (Santa Cruz) were transfected with TransIT®-293 Transfection Reagent (Mirus) to produce FUS KO HEK cell lines.

As the confirmation of FUS gene knockout, the presence or absence of the expression of FUS mRNA was confirmed using RT-PCR (SuperScript® IV One-Step RT-PCR System with ezDNase®, invitrogen, #12595100). The primer set used is shown in Table 10.

The total RNA was prepared using RNeasy Plus Mini Kit (QIAGEN). 1 μL of 10×ezDNase Buffer, 1 μL of ezDNase Enzyme, 1 μL of Template RNA (500 ng/μL), and 7 μL of water were mixed, and the digestion of gDNA was carried out at 37° C. for 5 minutes. Besides, RT-PCR was performed by mixing 10 μL of Template RNA (Digested gDNA), 25 μL of 2× Platinum SuperFi RT-PCR Master Mix, 2.5 μL of Primer Set I Mixture (each 10 μM), 2.5 μL of Primer Set V Mixture (each 10 μM), 0.5 μL of SuperScript IV RT Mix, and 9.5 μL of water, performing 40 cycles of reactions at 60° C. for 10 minutes, at 98° C. for 2 minutes, further at 98° C. for 10 seconds, at 62° C. for 10 seconds, and at 72° C. for 1 minute, followed by reaction at 72° C. for 5 minutes.

TABLE 10

| Name of | Name of | | SEQ ID |
|---|---|---|---|
| PCR | primer | Sequence | NO |
| Primer | (RT)Human | CACCAACTG | 260 |
| set I | beta | GGACGACAT | |
| | Actin-F | | |
| | (RT)Human | ACAGCCTGG | 261 |
| | beta | ATAGCAACG | |
| | Actin-R | | |
| Primer | (RT)Human | CTTATGGCC | 262 |
| set V | FUS-F_3 | AGAGCCAGA | |
| | | ACA | |
| | (RT)Human | ATCATGGGC | 263 |
| | FUS-R_3 | TGTCCCGTT | |
| | | TT | |

### Example 8

Production of co-expressed HEK293 cell lines of TurboGFP-fused FUS_{wild-type} and TurboFP635-fused FUS_{P525L}

TurboGFP-fused FUS_{wild-type} cDNA and TurboFP635-fused FUS_{P525L} cDNA were cloned into a multi-cloning site of pAAVS1-puro-DNR (Origene). pAAVS1-puro-DNR (Origene)_TurboGFP-FUS_{wild-type}, pAAVS1-puro-DNR (Origene)_TurboFP635-FUS_{P525L}, and pCas-Guide-AAVS1 (Origene) were transfected into previously prepared FUS KO HEK293 cells to produce the cells that co-express the TurboGFP-fused FUS_{wild-type} and TurboFP635-fused FUS_{P525L}.

The cell lines were cloned using On-Chip Sort (On-Chip Biotechnologies Co., Ltd.) by sorting the double positive cells of TurboGFP and TurboFP635, and then fractionating and culturing the single cells in 384-plates using On-chip SPiS (On-chip Biotechnologies Co., Ltd.).

### Example 9

Evaluation of RNA Interference Using Co-Expressed HEK293 Cell Lines of TurboGFP-Fused FUS_{wild-type} and TurboFP635-Fused FUS_{P525L}

25 μL of a mixed solution consisting of 25 μL of Opti-MEM (Invitrogen) and 1.5 μL of Lipofectamine® RNAi MAX (Invitrogen) and 25 μL of a mixed solution consisting of 25 μL of Opti-MEM (Invitrogen) and 0.5 μL of 10 μM siRNA were mixed and incubated at room temperature for 15 to 20 minutes.

Co-expressed HEK293 cell lines of TurboGFP-fused FUS_{wild-type} and TurboFP635-fused FUS_{P525L} were suspended in a warmed medium (FluoBrite™ DMENM containing 5% FBS, and the same below) so that the concentration was 3.0×10⁵ cells/mL. 100 μL of the cell suspension was mixed with 10 μL of the previously prepared Lipofectamine-siRNA complex, seeded in a CellCarer Ultra, collagen-coated 96-well plate, and cultured under the conditions of 37° C. and 5% CO₂ (hereinafter, cultured under the same conditions). 24 hours after the transfection, 100 μL of the medium was added. 48 hours after the transfection, 100 μL of the medium in each well was removed and then 100 μL of a solution containing 50 drops of Live Ready Probe® Reagent, Hoechst 33342 (Invitrogen) was added to 50 mL of 10% neutrally buffered formalin solution, (Sigma-Aldrich) and mixed to immobilize and stain the nucleus.

After incubation at 37° C. for about 30 minutes, data were acquired with an Operetta CLS® High Content Confocal Imaging System (PerkinElmer) equipped with a 20-fold immersion lens at confocal mode. The images are shown in FIG. 7. From the imagery data acquired, the total cell count (number of nucleus), the TurboGFP-positive cell count, and the TurboFP635-positive cell count were counted and the TurboGFP-positive cell rate (TurboGFP-positive cell count/total cell count) and the TurboFP635-positive cell rate (TurboFP635-positive cell count/total cell count) were calculated.

The TurboGFP-positive cells and the TurboFP635-positive cells were defined as follows:

### <TurboGFP-Positive Cells (FUS_{wild-type}-Expressing Cells)>

They were defined as the cells wherein the value obtained by dividing the total fluorescence intensity of TurboGFP in the nuclear region by the area (pixels) of the nuclear region is greater than or equal to 400.

### <TurboFP635-Positive Cells (FUS_{P525L}-Expressing Cells)>

They were defined as the cells wherein the value obtained by dividing the total fluorescence intensity of TurboFP635 in the cytoplasmic region by the area (pixels) of the cytoplasmic region is greater than or equal to 400.

Each positive cell rate was substituted into the following formula to calculate the relative values of the expression rate and the silencing rate.

$$( Expression Rate ) = 100 \times \{ ( Turbo GFP - positive cell rate after treatment of various siRNAs ( siRNA 001 to siRNA 060 ) ) - ( Turbo GFP - positive cell rate after trearment of positive control siRNA ) \} \text{} / \{ ( Turbo GFP - positive cell rate after treatment of negative control siRNA ) - ( Turbo GFP - positive cell rate after treatment of positive control siRNA ) \}$$

$$( Silencing Rate ) = 100 \times \{ ( Turbo GFP - positive - cell rate after treatment of various siRNAs ( siRNA 001 to siRNA 060 ) ) - ( Turbo GFP - positive cell rate after trearment of negative control siRNA ) \} \text{} / \{ ( Turbo GFP - positive cell rate after treatment of positive control siRNA ) - ( Turbo GFP - positive cell rate after treatment of negative control siRNA ) \}$$

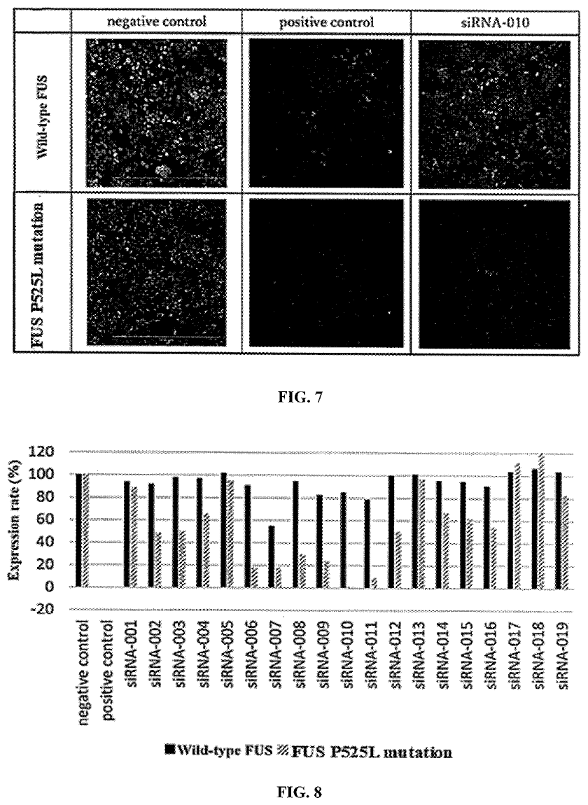

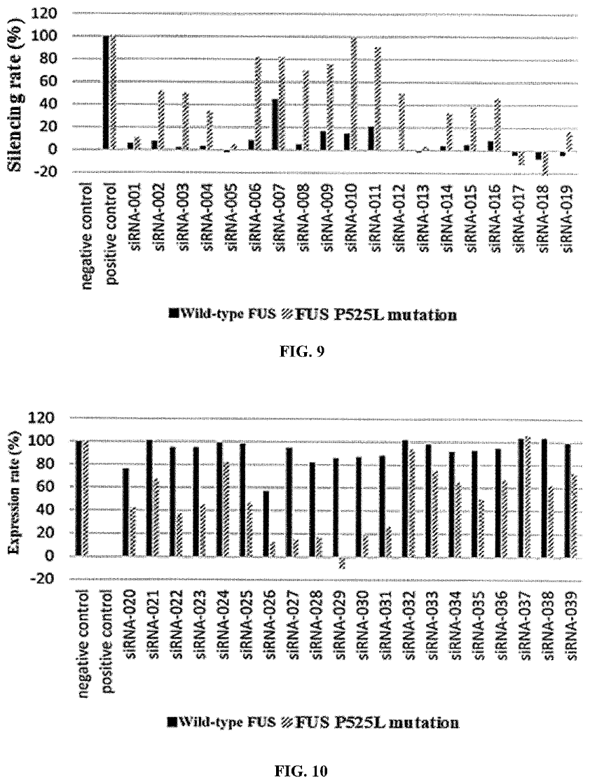

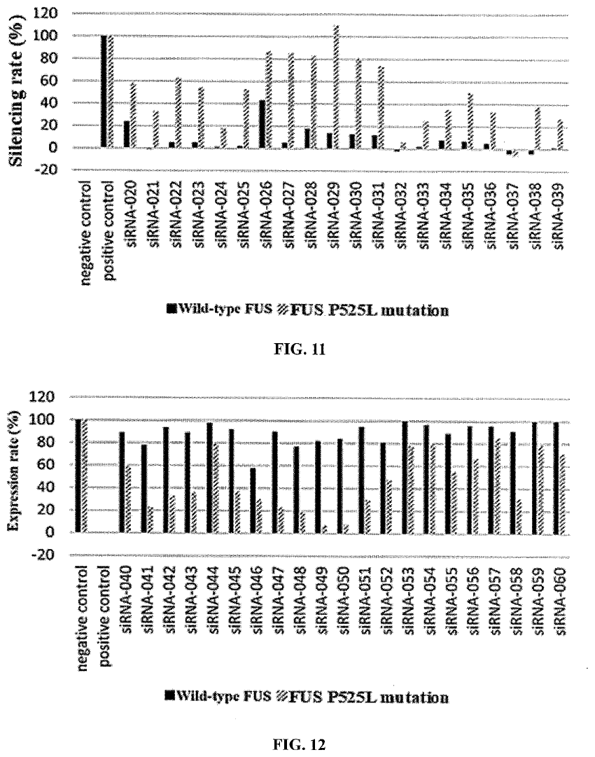

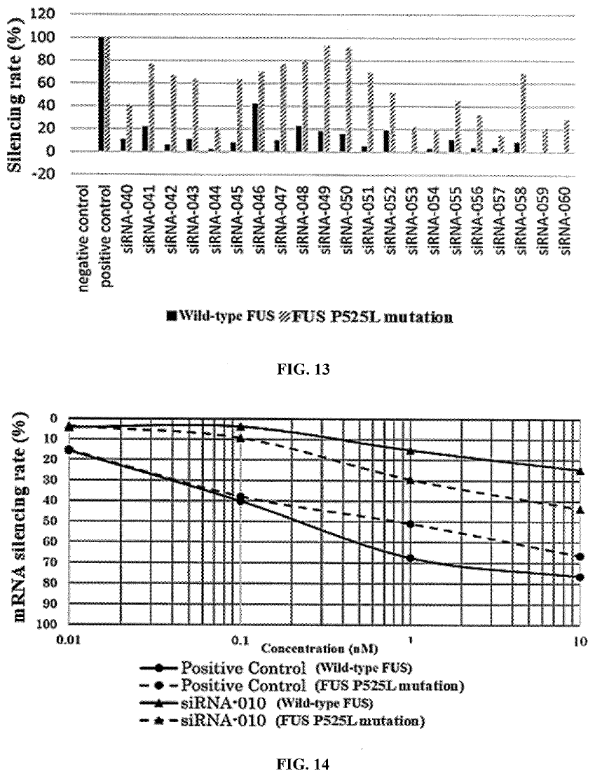

The expression rates are shown in FIGS. 8, 10 and 12. The silencing rates are shown in FIGS. 9, 11 and 13.

It was confirmed from the results of FIGS. 8 to 13 that the siRNAs of the present invention (siRNA-002, 003, 004, 006, 007, 008, 009, 010, 011, 012, 014, 015, 016, 019, 020, 021, 022, 023, 025, 026, 027, 028, 029, 030, 031, 033, 034, 035, 036, 038, 039, 040, 041, 042, 043, 045, 046, 047, 048, 049, 050, 051, 052, 053, 055, 056, 058, 059, 060) selectively exhibited silencing of FUS P525L mutation over the expression of wild-type FUS when co-expressed HEK cell lines were used. In either system of HEK cell lines co-expressed with HEK cell lines that express wild-type FUS and FUS P525L mutation separately, representative siRNAs that selectively exhibit silencing of FUS P525L mutation over the expression of wild-type FUS are listed below. The first representative examples include siRNA-002, 003, 004, 006, 008, 009, 010, 011, 012, 014, 016, 020, 021, 022, 023, 025, 027, 028, 029, 030, 031, 036, 045, 048, 049. The second representative examples include siRNA-002, 003, 006, 008, 009, 010, 011, 023, 025, 027, 028, 029, 030, 045, 049.

Therefore, it is particularly expected that the siRNAs composed of an antisense strand comprising a sequence identical or substantially identical to SEQ ID NO: 2, SEQ ID NO: 4, SEQ ID NO: 6, SEQ ID NO: 8, SEQ ID NO: 12, SEQ ID NO: 16, SEQ ID NO: 18, SEQ ID NO: 20, SEQ ID NO: 22, SEQ ID NO: 24, SEQ ID NO: 28, or SEQ ID NO: 32 excluding the overhang (dTdT) from the antisense strand of the above siRNAs, and the siRNAs composed of an antisense strand comprising a sequence identical or substantially identical to SEQ ID NO: 4, SEQ ID NO: 6, SEQ ID NO: 12, SEQ ID NO: 16, SEQ ID NO: 18, SEQ ID NO: 20, or SEQ ID NO: 22 excluding the overhang (dTdT) from the antisense strand of the above siRNAs, as the siRNAs of the present invention, will be useful as therapeutic agents for the ALS associated with the FUS P525L mutation gene.

### Example 10

### Confirmation of the Concentration-Dependent Inhibitory Effect on the Expression of FUS Genes by Real-Time PCR

25 μL of a mixed solution consisting of 25 μL of Opti-MEM (Invitrogen) and 1.5 μL of Lipofectamine® RNAi MAX (Invitrogen), and 25 μL of a mixed solution consisting of 25 μL of Opti-MEM (Invitrogen) and 0.5 μL of siRNA (0.01 μM, 0.1 μM, 1 μM, 10 μM) were mixed and incubated at room temperature for 15 to 20 minutes. Then, transfection of siRNA into cells was performed according to the method indicated in “Evaluation of RNA interference using co-expressed HEK293 cell lines of TurboGFP-fused FUSwild-type and TurboFP635-fused FUSP525L”.

48 hours after the transfection, the medium was removed completely, and 50 μL of a cell lysate, which was a mixture of 0.5 μL of DNase I (Life Technologies Japan) and 49.5 μL of Lysis Solution (Life Technologies Japan), was added and incubated at room temperature for 5 minutes. After that, 5 μL of Stop Solution (Life Technologies Japan) was added and mixed, and then incubated at room temperature for 2 minutes, which was subjected to a reverse transcription reaction.

To a reaction solution of the reverse transcription, which was a mixture of 25 μL of 2× Fast Advanced RT Buffer (Life Technologies Japan), 2.5 μL of 20× Fast Advanced RT Enzyme Mix (Life Technologies Japan), and 12.5 μL of Nuclease-free water, 10 μL of the previously prepared cell lysate was added, and reacted at 37° C. for 30 minutes and then at 95° C. for 5 minutes to synthesize the cDNA.

In the real-time PCR, three genes, i.e., TurboGFP-fused FUS_{wild-type} gene, TurboFP635-fused FUS_{P525L} gene, and endogenous control gene (GAPDH) were detected in the same reaction system.

The reaction solution was prepared by mixing 10 μL of TaqMan® Fast Advanced Master Mix (Life Technologies Japan), 0.06 μL each of 100 μM primer (GFP_X_F, GFP_X_R, FP635_X_F, FP635_X_R), 0.5 μL each of 10 μM TaqMan® probe (TurboGFP (NED), TurboFP635 (FAM), 1.0 μL of 20× TaqMan® Assay (GAPDH) (Life Technologies Japan), and 4 μL of the previously prepared cDNA. The reaction solution was allowed to react in a real-time PCR equipment (QuantStudio 3, Life Technologies Japan) at 50° C. for 2 minutes and then at 95° C. for 20 seconds, followed by 40 cycles of reactions at 95° C. for 1 second and then at 60° C. for 20 seconds.

The primers and TaqMan probes used in the real-time PCR are shown in Tables 11 and 12.

The amount of gene expression was calculated as relative values according to Δδct method. Here, the expression rate was calculated by taking Mock Transfection as 100%. The silencing rate (=100−expression rate) was calculated from the expression rate.

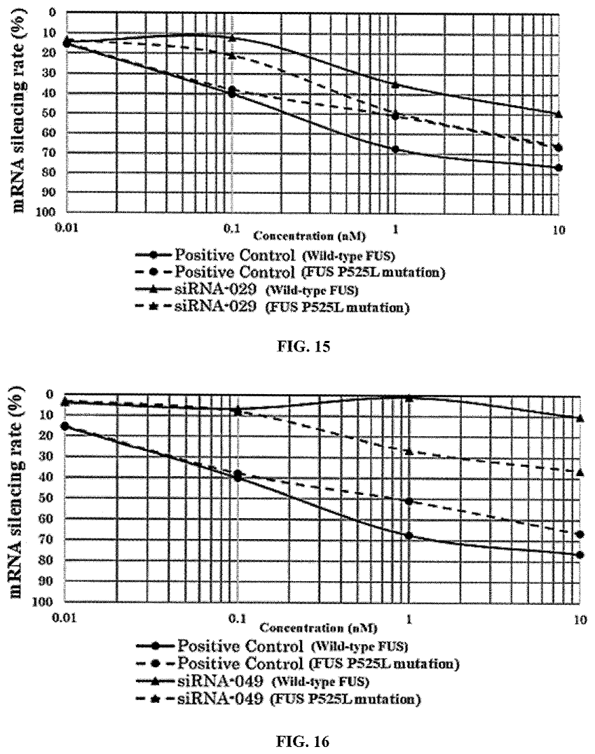

The results of siRNA-010, siRNA-029, and siRNA-049 are shown in FIGS. 14 to 16, respectively.

TABLE 11

| Name of | | SEQ ID |
|---|---|---|
| primer | Sequence | NO |
| GFP_X_F | TGGGCATCGT | 264 |
| | GGAGTACCA | |
| GFP_X_R | TGCTTGTTGG | 265 |
| | GTATAATCGT | |
| | TTGA | |
| FP635_X_F | CTGCGACCTG | 266 |
| | CCTAGCAAAC | |
| FP635_X_R | CCATAGCTTT | 267 |
| | GGGTTGCTTG | |
| | TT | |

TABLE 12

| Name of probe | Sequence | SEQ ID NO |
|---|---|---|
| TurboGFP(NED) | AGACCCCGGATGCAG | 268 |
| TurboFP635(FAM) | ACAGCTCCGGACTCA | 269 |

The IC₅₀ of each siRNA was calculated from the results of FIGS. 14 to 16 with a four-parameter fit model using XLFit, as shown in Table 13. It is suggested from the comparison of the IC₅₀ to the wild-type FUS with the IC₅₀ to the FUS P525L mutation that siRNA-010, siRNA-029, and siRNA-049 exhibit silencing of FUS P525L mutation with higher selectivity than wild-type FUS.

TABLE 13

| | IC₅₀ to | IC₅₀ to FUS |
|---|---|---|
| | wild-type FUS | P525L mutation |
| Positive Control | 0.3 nM | 0.9 nM |
| siRNA-010 | >10 nM | >10 nM |
| siRNA-029 | >10 nM | 1.6 nM |
| siRNA-049 | >10 nM | >10 nM |

### Example 11

### Confirmation of the Time-Dependent Inhibitory Effect on the Expression of FUS Genes by Real-Time PCR

Transfection of siRNA (10 nM) was performed according to the method indicated in the “Evaluation of RNA interference using co-expressed HEK293 cell lines of TurboGFP-fused FUS_{wild-type} and TurboFP635-fused FUS_{P525L}”. 12, 24, 48, and 72 hours after the transfection, the medium was removed completely and cryopreserved (at −80° C. or lower) until measurement was performed.

Each cryopreserved sample was analyzed for the amount of gene expression according to the method indicated in “Confirmation of the concentration-dependent inhibitory effect on the expression of FUS genes by real-time PCR”. The primers and TaqMan probes used in the real-time PCR are shown in Tables 11 and 12.

The amount of gene expression was calculated as relative values according to Δδct method. Here, the expression rate was calculated by taking the expression amount in the negative control siRNA as 100%. The silencing rate (=100−expression rate) was calculated from the expression rate.

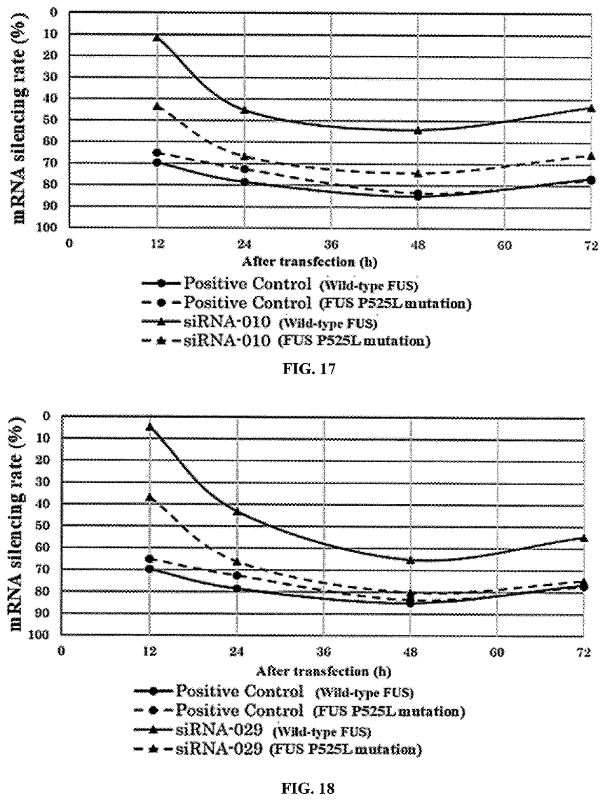

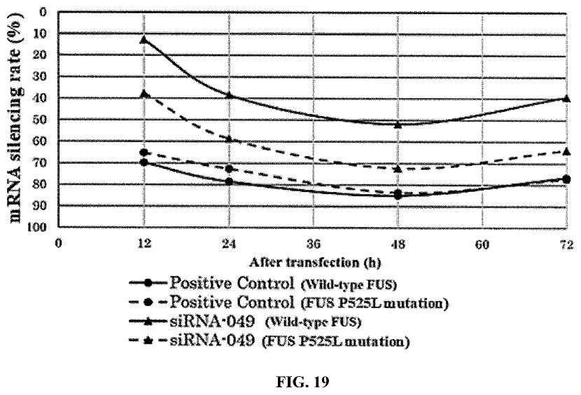

The results of siRNA-010, siRNA-029, and siRNA-049 are shown in FIGS. 17 to 19, respectively.

It is suggested from the results of FIGS. 17 to 19 that siRNA-010, siRNA-029, and siRNA-049 exhibit silencing of FUS P525L mutation with higher selectivity than wild-type FUS at any time after the transfection.

[Table of sequence listing] 202108 Morita Ohara—Sequence List.txt

1. An siRNA comprising a sense strand and an antisense strand, wherein the antisense strand comprises a region complementary or substantially complementary to a portion of the mRNA encoding FUS P525L mutation, and wherein the complementary region is 19 to 21 nucleotides in length.

2. The siRNA of claim 1, where the antisense strand comprises a sequence identical or substantially identical to SEQ ID NO: 2, SEQ ID NO: 4, SEQ ID NO: 6, SEQ ID NO: 8, SEQ ID NO: 10, SEQ ID NO: 12, SEQ ID NO: 16, SEQ ID NO: 18, SEQ ID NO: 20, SEQ ID NO: 22, SEQ ID NO: 24, SEQ ID NO: 28, or SEQ ID NO: 32.

3. The siRNA of claim 1, wherein the antisense strand comprises a sequence identical or substantially identical to SEQ ID NO: 2, SEQ ID NO: 4, SEQ ID NO: 6, SEQ ID NO: 8, SEQ ID NO: 10, SEQ ID NO: 12, SEQ ID NO: 16, SEQ ID NO: 18, SEQ ID NO: 20, SEQ ID NO: 22, or SEQ ID NO: 32.

4. The siRNA of claim 1, wherein the antisense strand comprises a sequence identical or substantially identical to SEQ ID NO: 2, SEQ ID NO: 4, SEQ ID NO: 6, SEQ ID NO: 8, SEQ ID NO: 12, SEQ ID NO: 16, SEQ ID NO: 18, SEQ ID NO: 20, SEQ ID NO: 22, SEQ ID NO: 24, SEQ ID NO: 28, or SEQ ID NO: 32.

5. The siRNA of claim 1, wherein the antisense strand comprises a sequence identical or substantially identical to SEQ ID NO: 4, SEQ ID NO: 6, SEQ ID NO: 12, SEQ ID NO: 16, SEQ ID NO: 18, SEQ ID NO: 20, or SEQ ID NO: 22.

6. The siRNA of claim 1, comprising a sequence identical or substantially identical to
double-stranded RNA consisting of the sense strand of SEQ ID NO: 1 and the antisense strand of SEQ ID NO: 2,
double-stranded RNA consisting of the sense strand of SEQ ID NO: 3 and the antisense strand of SEQ ID NO: 4,
double-stranded RNA consisting of the sense strand of SEQ ID NO: 5 and the antisense strand of SEQ ID NO: 6,
double-stranded RNA consisting of the sense strand of SEQ ID NO: 7 and the antisense strand of SEQ ID NO: 8,
double-stranded RNA consisting of the sense strand of SEQ ID NO: 9 and the antisense strand of SEQ ID NO: 10,
double-stranded RNA consisting of the sense strand of SEQ ID NO: 11 and the antisense strand of SEQ ID NO: 12,
double-stranded RNA consisting of the sense strand of SEQ ID NO: 15 and the antisense strand of SEQ ID NO: 16,
double-stranded RNA consisting of the sense strand of SEQ ID NO: 17 and the antisense strand of SEQ ID NO: 18,
double-stranded RNA consisting of the sense strand of SEQ ID NO: 19 and the antisense strand of SEQ ID NO: 20,
double-stranded RNA consisting of the sense strand of SEQ ID NO: 21 and the antisense strand of SEQ ID NO: 22,
double-stranded RNA consisting of the sense strand of SEQ ID NO: 23 and the antisense strand of SEQ ID NO: 24,
double-stranded RNA consisting of the sense strand of SEQ ID NO: 27 and the antisense strand of SEQ ID NO: 28,
double-stranded RNA consisting of the sense strand of SEQ ID NO: 31 and the antisense strand of SEQ ID NO: 32,
double-stranded RNA consisting of the sense strand of SEQ ID NO: 39 and the antisense strand of SEQ ID NO: 40,
double-stranded RNA consisting of the sense strand of SEQ ID NO: 41 and the antisense strand of SEQ ID NO: 42,
double-stranded RNA consisting of the sense strand of SEQ ID NO: 43 and the antisense strand of SEQ ID NO: 44,
double-stranded RNA consisting of the sense strand of SEQ ID NO: 45 and the antisense strand of SEQ ID NO: 46,
double-stranded RNA consisting of the sense strand of SEQ ID NO: 47 and the antisense strand of SEQ ID NO: 48,
double-stranded RNA consisting of the sense strand of SEQ ID NO: 49 and the antisense strand of SEQ ID NO: 50,
double-stranded RNA consisting of the sense strand of SEQ ID NO: 53 and the antisense strand of SEQ ID NO: 54,
double-stranded RNA consisting of the sense strand of SEQ ID NO: 55 and the antisense strand of SEQ ID NO: 56,
double-stranded RNA consisting of the sense strand of SEQ ID NO: 57 and the antisense strand of SEQ ID NO: 58,
double-stranded RNA consisting of the sense strand of SEQ ID NO: 59 and the antisense strand of SEQ ID NO: 60,
double-stranded RNA consisting of the sense strand of SEQ ID NO: 61 and the antisense strand of SEQ ID NO: 62,
double-stranded RNA consisting of the sense strand of SEQ ID NO: 71 and the antisense strand of SEQ ID NO: 72,
double-stranded RNA consisting of the sense strand of SEQ ID NO: 89 and the antisense strand of SEQ ID NO: 90,
double-stranded RNA consisting of the sense strand of SEQ ID NO: 95 and the antisense strand of SEQ ID NO: 96,
double-stranded RNA composed of the sense strand of SEQ ID NO: 97 and the antisense strand of SEQ ID NO: 98, or
double-stranded RNA consisting of the sense strand of SEQ ID NO: 113 and the antisense strand of SEQ ID NO: 114.

7. The siRNA of claim 1, comprising a sequence identical or substantially identical to
double-stranded RNA consisting of the sense strand of SEQ ID NO: 1 and the antisense strand of SEQ ID NO: 2,
double-stranded RNA consisting of the sense strand of SEQ ID NO: 3 and the antisense strand of SEQ ID NO: 4,
double-stranded RNA consisting of the sense strand of SEQ ID NO: 5 and the antisense strand of SEQ ID NO: 6,
double-stranded RNA consisting of the sense strand of SEQ ID NO: 7 and the antisense strand of SEQ ID NO: 8,
double-stranded RNA consisting of the sense strand of SEQ ID NO: 9 and the antisense strand of SEQ ID NO: 10,
double-stranded RNA consisting of the sense strand of SEQ ID NO: 11 and the antisense strand of SEQ ID NO: 12,
double-stranded RNA consisting of the sense strand of SEQ ID NO: 15 and the antisense strand of SEQ ID NO: 16,
double-stranded RNA consisting of the sense strand of SEQ ID NO: 17 and the antisense strand of SEQ ID NO: 18,
double-stranded RNA consisting of the sense strand of SEQ ID NO: 19 and the antisense strand of SEQ ID NO: 20,
double-stranded RNA consisting of the sense strand of SEQ ID NO: 21 and the antisense strand of SEQ ID NO: 22,
double-stranded RNA consisting of the sense strand of SEQ ID NO: 45 and the antisense strand of SEQ ID NO: 46,
double-stranded RNA consisting of the sense strand of SEQ ID NO: 47 and the antisense strand of SEQ ID NO: 48,
double-stranded RNA consisting of the sense strand of SEQ ID NO: 49 and the antisense strand of SEQ ID NO: 50,
double-stranded RNA consisting of the sense strand of SEQ ID NO: 53 and the antisense strand of SEQ ID NO: 54,
double-stranded RNA consisting of the sense strand of SEQ ID NO: 55 and the antisense strand of SEQ ID NO: 56,
double-stranded RNA consisting of the sense strand of SEQ ID NO: 57 and the antisense strand of SEQ ID NO: 58,
double-stranded RNA consisting of the sense strand of SEQ ID NO: 59 and the antisense strand of SEQ ID NO: 60,
double-stranded RNA consisting of the sense strand of SEQ ID NO: 89 and the antisense strand of SEQ ID NO: 90,
double-stranded RNA consisting of the sense strand of SEQ ID NO: 97 and the antisense strand of SEQ ID NO: 98, or
double-stranded RNA consisting of the sense strand of SEQ ID NO: 113 and the antisense strand of SEQ ID NO: 114.

8. The siRNA of claim 1, comprising a sequence identical or substantially identical to
double-stranded RNA consisting of the sense strand of SEQ ID NO: 3 and the antisense strand of SEQ ID NO: 4,
double-stranded RNA consisting of the sense strand of SEQ ID NO: 5 and the antisense strand of SEQ ID NO: 6,
double-stranded RNA consisting of the sense strand of SEQ ID NO: 7 and the antisense strand of SEQ ID NO: 8,
double-stranded RNA consisting of the sense strand of SEQ ID NO: 11 and the antisense strand of SEQ ID NO: 12,
double-stranded RNA consisting of the sense strand of SEQ ID NO: 15 and the antisense strand of SEQ ID NO: 16,
double-stranded RNA consisting of the sense strand of SEQ ID NO: 17 and the antisense strand of SEQ ID NO: 18,
double-stranded RNA consisting of the sense strand of SEQ ID NO: 19 and the antisense strand of SEQ ID NO: 20,
double-stranded RNA consisting of the sense strand of SEQ ID NO: 21 and the antisense strand of SEQ ID NO: 22,
double-stranded RNA consisting of the sense strand of SEQ ID NO: 23 and the antisense strand of SEQ ID NO: 24,
double-stranded RNA consisting of the sense strand of SEQ ID NO: 27 and the antisense strand of SEQ ID NO: 28,
double-stranded RNA consisting of the sense strand of SEQ ID NO: 31 and the antisense strand of SEQ ID NO: 32,
double-stranded RNA consisting of the sense strand of SEQ ID NO: 39 and the antisense strand of SEQ ID NO: 40,
double-stranded RNA consisting of the sense strand of SEQ ID NO: 41 and the antisense strand of SEQ ID NO: 42,
double-stranded RNA consisting of the sense strand of SEQ ID NO: 43 and the antisense strand of SEQ ID NO: 44,
double-stranded RNA consisting of the sense strand of SEQ ID NO: 45 and the antisense strand of SEQ ID NO: 46,
double-stranded RNA consisting of the sense strand of SEQ ID NO: 49 and the antisense strand of SEQ ID NO: 50,
double-stranded RNA consisting of the sense strand of SEQ ID NO: 53 and the antisense strand of SEQ ID NO: 54,
double-stranded RNA consisting of the sense strand of SEQ ID NO: 55 and the antisense strand of SEQ ID NO: 56,
double-stranded RNA consisting of the sense strand of SEQ ID NO: 57 and the antisense strand of SEQ ID NO: 58,
double-stranded RNA consisting of the sense strand of SEQ ID NO: 59 and the antisense strand of SEQ ID NO: 60,
double-stranded RNA consisting of the sense strand of SEQ ID NO: 61 and the antisense strand of SEQ ID NO: 62,
double-stranded RNA consisting of the sense strand of SEQ ID NO: 71 and the antisense strand of SEQ ID NO: 72,
double-stranded RNA consisting of the sense strand of SEQ ID NO: 89 and the antisense strand of SEQ ID NO: 90,
double-stranded RNA consisting of the sense strand of SEQ ID NO: 95 and the antisense strand of SEQ ID NO: 96, or
double-stranded RNA consisting of the sense strand of SEQ ID NO: 97 and the antisense strand of SEQ ID NO: 98.

9. The siRNA of claim 1, comprising a sequence identical or substantially identical to
double-stranded RNA consisting of the sense strand of SEQ ID NO: 3 and the antisense strand of SEQ ID NO: 4,
double-stranded RNA consisting of the sense strand of SEQ ID NO: 5 and the antisense strand of SEQ ID NO: 6,
double-stranded RNA consisting of the sense strand of SEQ ID NO: 11 and the antisense strand of SEQ ID NO: 12,
double-stranded RNA consisting of the sense strand of SEQ ID NO: 15 and the antisense strand of SEQ ID NO: 16,
double-stranded RNA consisting of the sense strand of SEQ ID NO: 17 and the antisense strand of SEQ ID NO: 18,
double-stranded RNA consisting of the sense strand of SEQ ID NO: 19 and the antisense strand of SEQ ID NO: 20,
double-stranded RNA consisting of the sense strand of SEQ ID NO: 21 and the antisense strand of SEQ ID NO: 22,
double-stranded RNA consisting of the sense strand of SEQ ID NO: 45 and the antisense strand of SEQ ID NO: 46,
double-stranded RNA consisting of the sense strand of SEQ ID NO: 49 and the antisense strand of SEQ ID NO: 50,
double-stranded RNA consisting of the sense strand of SEQ ID NO: 53 and the antisense strand of SEQ ID NO: 54,
double-stranded RNA consisting of the sense strand of SEQ ID NO: 55 and the antisense strand of SEQ ID NO: 56,
double-stranded RNA consisting of the sense strand of SEQ ID NO: 57 and the antisense strand of SEQ ID NO: 58,
double-stranded RNA consisting of the sense strand of SEQ ID NO: 59 and the antisense strand of SEQ ID NO: 60,
double-stranded RNA consisting of the sense strand of SEQ ID NO: 89 and the antisense strand of SEQ ID NO: 90, or
double-stranded RNA consisting of the sense strand of SEQ ID NO: 97 and the antisense strand of SEQ ID NO: 98.

10. The siRNA of claim 1, wherein the siRNA comprises an overhang of 2 nucleotides in length at 3′ end of its sense strand and/or antisense strand and is a 21 to 23 base pair.

11. The siRNA of claim 1, wherein the siRNA comprises at least one modified nucleotide.

12. The siRNA of claim 11, wherein the at least one modified nucleotide comprises a 5′-phosphorothioate group.

13. The siRNA of claim 11, wherein the at least one modified nucleotide is selected from the group consisting of 2′-deoxy-modified nucleotide, 2′-deoxy-2′-fluoro-modified nucleotide, 2′-O-methyl modified nucleotide, 2′-O-methoxyethyl modified nucleotide, and the nucleotide wherein the 2′-O atom and 4′-C atom are crosslinked via a methylene or ethylene group.

14. The siRNA of claim 1 for silencing of FUS P525L mutation.

15. The siRNA of claim 1 for selectively silencing of FUS P525L mutation without substantially silencing of wild-type FUS.

16. A pharmaceutical composition comprising the siRNA of claim 1.

17. A pharmaceutical composition comprising the siRNA of any of claim 1, for silencing of FUS P525L mutation.

18. A pharmaceutical composition comprising the siRNA of claim 1, for selectively silencing of FUS P525L mutation without substantially silencing of wild-type FUS.

19. A FUS P525L mutation expression inhibitor comprising the siRNA of claim 1.

20. An ALS therapeutic agent comprising the siRNA of claim 1 or a pharmaceutical composition thereof.

21. An ALS therapeutic agent of claim 20, wherein the ALS is juvenile ALS with P525L mutation in FUS gene.

22. A method of treating ALS characterized by administering an effective amount of the siRNA of claim 1.

23. (canceled)

24. A DNA vector for expressing the siRNA of claim 1 in cells.

25. A pharmaceutical composition comprising the DNA vector of claim 24 and a pharmaceutically acceptable carrier.

26. A Cell comprising the DNA vector of claim 24.

27. The cell of claim 26, wherein the cell is a mammalian cell.

28. A method of screening an ALS therapeutic agent that selectively exhibits silencing of FUS P525L mutation, characterized by determining a silencing rate of wild-type FUS and a silencing rate of FUS P525L mutation.

29. A method of screening siRNA for use as an active ingredient of an ALS therapeutic agent, comprising
(1) a step of designing the siRNAs comprising a region complementary or substantially complementary to a portion of mRNA encoding FUS P525L mutation, wherein the complementary region is 19 to 21 nucleotides in length,
(2) a step of producing the siRNAs designed in step (1), and
(3) a step of screening for the siRNA that selectively exhibits silencing of FUS P525L mutation without substantially silencing of wild-type FUS, from the siRNAs produced in step (2).

30. A method of producing an ALS therapeutic agent comprising the siRNA that selectively exhibits silencing of FUS P525L mutation as an active ingredient, comprising
(1) a step of designing the siRNAs comprising a region complementary or substantially complementary to a portion of mRNA encoding FUS P525L mutation, wherein the complementary region is 19 to 21 nucleotides in length,
(2) a step of producing the siRNAs designed in step (1),
(3) a step of screening for the siRNA that selectively exhibits silencing of FUS P525L mutation without substantially silencing of wild-type FUS, from the siRNAs produced in step (2),
(4) a step of producing a pharmaceutical composition containing the siRNA screened in step (3) as an active ingredient, and
(5) a step of confirming the effect of the pharmaceutical composition produced in step (4) as an ALS therapeutic agent.


SEQUENCE LISTING (269 sequences)

SEQ ID NO: 1 - RNA, 19 nt, Artificial Sequence
  siRNA sequence
aggaucgcagggagaggcu

SEQ ID NO: 2 - RNA, 19 nt, Artificial Sequence
  siRNA sequence
agccucucccugcgauccu

SEQ ID NO: 3 - RNA, 19 nt, Artificial Sequence
  siRNA sequence
ggaucgcagggagaggcug

SEQ ID NO: 4 - RNA, 19 nt, Artificial Sequence
  siRNA sequence
cagccucucccugcgaucc

SEQ ID NO: 5 - RNA, 19 nt, Artificial Sequence
  siRNA sequence
gaucgcagggagaggcugu

SEQ ID NO: 6 - RNA, 19 nt, Artificial Sequence
  siRNA sequence
acagccucucccugcgauc

SEQ ID NO: 7 - RNA, 19 nt, Artificial Sequence
  siRNA sequence
aucgcagggagaggcugua

SEQ ID NO: 8 - RNA, 19 nt, Artificial Sequence
  siRNA sequence
uacagccucucccugcgau

SEQ ID NO: 9 - RNA, 19 nt, Artificial Sequence
  siRNA sequence
ucgcagggagaggcuguau

SEQ ID NO: 10 - RNA, 19 nt, Artificial Sequence
  siRNA sequence
auacagccucucccugcga

SEQ ID NO: 11 - RNA, 19 nt, Artificial Sequence
  siRNA sequence
cgcagggagaggcuguauu

SEQ ID NO: 12 - RNA, 19 nt, Artificial Sequence
  siRNA sequence
aauacagccucucccugcg

SEQ ID NO: 13 - RNA, 19 nt, Artificial Sequence
  siRNA sequence
gcagggagaggcuguauua

SEQ ID NO: 14 - RNA, 19 nt, Artificial Sequence
  siRNA sequence
uaauacagccucucccugc

SEQ ID NO: 15 - RNA, 19 nt, Artificial Sequence
  siRNA sequence
cagggagaggcuguauuaa

SEQ ID NO: 16 - RNA, 19 nt, Artificial Sequence
  siRNA sequence
uuaauacagccucucccug

SEQ ID NO: 17 - RNA, 19 nt, Artificial Sequence
  siRNA sequence
agggagaggcuguauuaau

SEQ ID NO: 18 - RNA, 19 nt, Artificial Sequence
  siRNA sequence
auuaauacagccucucccu

SEQ ID NO: 19 - RNA, 19 nt, Artificial Sequence
  siRNA sequence
gggagaggcuguauuaauu

SEQ ID NO: 20 - RNA, 19 nt, Artificial Sequence
  siRNA sequence
aauuaauacagccucuccc

SEQ ID NO: 21 - RNA, 19 nt, Artificial Sequence
  siRNA sequence
ggagaggcuguauuaauua

SEQ ID NO: 22 - RNA, 19 nt, Artificial Sequence
  siRNA sequence
uaauuaauacagccucucc

SEQ ID NO: 23 - RNA, 19 nt, Artificial Sequence
  siRNA sequence
gagaggcuguauuaauuag

SEQ ID NO: 24 - RNA, 19 nt, Artificial Sequence
  siRNA sequence
cuaauuaauacagccucuc

SEQ ID NO: 25 - RNA, 19 nt, Artificial Sequence
  siRNA sequence
agaggcuguauuaauuagc

SEQ ID NO: 26 - RNA, 19 nt, Artificial Sequence
  siRNA sequence
gcuaauuaauacagccucu

SEQ ID NO: 27 - RNA, 19 nt, Artificial Sequence
  siRNA sequence
gaggcuguauuaauuagcc

SEQ ID NO: 28 - RNA, 19 nt, Artificial Sequence
  siRNA sequence
ggcuaauuaauacagccuc

SEQ ID NO: 29 - RNA, 19 nt, Artificial Sequence
  siRNA sequence
aggcuguauuaauuagccu

SEQ ID NO: 30 - RNA, 19 nt, Artificial Sequence
  siRNA sequence
aggcuaauuaauacagccu

SEQ ID NO: 31 - RNA, 19 nt, Artificial Sequence
  siRNA sequence
ggcuguauuaauuagccug

SEQ ID NO: 32 - RNA, 19 nt, Artificial Sequence
  siRNA sequence
caggcuaauuaauacagcc

SEQ ID NO: 33 - RNA, 19 nt, Artificial Sequence
  siRNA sequence
gcuguauuaauuagccugg

SEQ ID NO: 34 - RNA, 19 nt, Artificial Sequence
  siRNA sequence
ccaggcuaauuaauacagc

SEQ ID NO: 35 - RNA, 19 nt, Artificial Sequence
  siRNA sequence
cuguauuaauuagccuggc

SEQ ID NO: 36 - RNA, 19 nt, Artificial Sequence
  siRNA sequence
gccaggcuaauuaauacag

SEQ ID NO: 37 - RNA, 19 nt, Artificial Sequence
  siRNA sequence
uguauuaauuagccuggcu

SEQ ID NO: 38 - RNA, 19 nt, Artificial Sequence
  siRNA sequence
agccaggcuaauuaauaca

SEQ ID NO: 39 - RNA, 20 nt, Artificial Sequence
  siRNA sequence
caggaucgcagggagaggcu

SEQ ID NO: 40 - RNA, 20 nt, Artificial Sequence
  siRNA sequence
agccucucccugcgauccug

SEQ ID NO: 41 - RNA, 20 nt, Artificial Sequence
  siRNA sequence
aggaucgcagggagaggcug

SEQ ID NO: 42 - RNA, 20 nt, Artificial Sequence
  siRNA sequence
cagccucucccugcgauccu

SEQ ID NO: 43 - RNA, 20 nt, Artificial Sequence
  siRNA sequence
ggaucgcagggagaggcugu

SEQ ID NO: 44 - RNA, 20 nt, Artificial Sequence
  siRNA sequence
acagccucucccugcgaucc

SEQ ID NO: 45 - RNA, 20 nt, Artificial Sequence
  siRNA sequence
gaucgcagggagaggcugua

SEQ ID NO: 46 - RNA, 20 nt, Artificial Sequence
  siRNA sequence
uacagccucucccugcgauc

SEQ ID NO: 47 - RNA, 20 nt, Artificial Sequence
  siRNA sequence
aucgcagggagaggcuguau

SEQ ID NO: 48 - RNA, 20 nt, Artificial Sequence
  siRNA sequence
auacagccucucccugcgau

SEQ ID NO: 49 - RNA, 20 nt, Artificial Sequence
  siRNA sequence
ucgcagggagaggcuguauu

SEQ ID NO: 50 - RNA, 20 nt, Artificial Sequence
  siRNA sequence
aauacagccucucccugcga

SEQ ID NO: 51 - RNA, 20 nt, Artificial Sequence
  siRNA sequence
cgcagggagaggcuguauua

SEQ ID NO: 52 - RNA, 20 nt, Artificial Sequence
  siRNA sequence
uaauacagccucucccugcg

SEQ ID NO: 53 - RNA, 20 nt, Artificial Sequence
  siRNA sequence
gcagggagaggcuguauuaa

SEQ ID NO: 54 - RNA, 20 nt, Artificial Sequence
  siRNA sequence
uuaauacagccucucccugc

SEQ ID NO: 55 - RNA, 20 nt, Artificial Sequence
  siRNA sequence
cagggagaggcuguauuaau

SEQ ID NO: 56 - RNA, 20 nt, Artificial Sequence
  siRNA sequence
auuaauacagccucucccug

SEQ ID NO: 57 - RNA, 20 nt, Artificial Sequence
  siRNA sequence
agggagaggcuguauuaauu

SEQ ID NO: 58 - RNA, 20 nt, Artificial Sequence
  siRNA sequence
aauuaauacagccucucccu

SEQ ID NO: 59 - RNA, 20 nt, Artificial Sequence
  siRNA sequence
gggagaggcuguauuaauua

SEQ ID NO: 60 - RNA, 20 nt, Artificial sequence
  siRNA sequence
uaauuaauacagccucuccc

SEQ ID NO: 61 - RNA, 20 nt, Artificial Sequence
  siRNA sequence
ggagaggcuguauuaauuag

SEQ ID NO: 62 - RNA, 20 nt, Artificial Sequence
  siRNA sequence
cuaauuaauacagccucucc

SEQ ID NO: 63 - RNA, 20 nt, Artificial Sequence
  siRNA sequence
gagaggcuguauuaauuagc

SEQ ID NO: 64 - RNA, 20 nt, Artificial Sequence
  siRNA sequence
gcuaauuaauacagccucuc

SEQ ID NO: 65 - RNA, 20 nt, Artificial Sequence
  siRNA sequence
agaggcuguauuaauuagcc

SEQ ID NO: 66 - RNA, 20 nt, Artificial Sequence
  siRNA sequence
ggcuaauuaauacagccucu

SEQ ID NO: 67 - RNA, 20 nt, Artificial Sequence
  siRNA sequence
gaggcuguauuaauuagccu

SEQ ID NO: 68 - RNA, 20 nt, Artificial Sequence
  siRNA sequence
aggcuaauuaauacagccuc

SEQ ID NO: 69 - RNA, 20 nt, Artificial Sequence
  siRNA sequence
aggcuguauuaauuagccug

SEQ ID NO: 70 - RNA, 20 nt, Artificial Sequence
  siRNA sequence
caggcuaauuaauacagccu

SEQ ID NO: 71 - RNA, 20 nt, Artificial Sequence
  siRNA sequence
ggcuguauuaauuagccugg

SEQ ID NO: 72 - RNA, 20 nt, Artificial Sequence
  siRNA sequence
ccaggcuaauuaauacagcc

SEQ ID NO: 73 - RNA, 20 nt, Artificial Sequence
  siRNA sequence
gcuguauuaauuagccuggc

SEQ ID NO: 74 - RNA, 20 nt, Artificial Sequence
  siRNA sequence
gccaggcuaauuaauacagc

SEQ ID NO: 75 - RNA, 20 nt, Artificial Sequence
  siRNA sequence
cuguauuaauuagccuggcu

SEQ ID NO: 76 - RNA, 20 nt, Artificial Sequence
  siRNA sequence
agccaggcuaauuaauacag

SEQ ID NO: 77 - RNA, 20 nt, Artificial Sequence
  siRNA sequence
uguauuaauuagccuggcuc

SEQ ID NO: 78 - RNA, 20 nt, Artificial Sequence
  siRNA sequence
gagccaggcuaauuaauaca

SEQ ID NO: 79 - RNA, 21 nt, Artificial Sequence
  siRNA sequence
acaggaucgcagggagaggcu

SEQ ID NO: 80 - RNA, 21 nt, Artificial Sequence
  siRNA sequence
agccucucccugcgauccugu

SEQ ID NO: 81 - RNA, 21 nt, Artificial Sequence
  siRNA sequence
caggaucgcagggagaggcug

SEQ ID NO: 82 - RNA, 21 nt, Artificial Sequence
  siRNA sequence
cagccucucccugcgauccug

SEQ ID NO: 83 - RNA, 21 nt, Artificial Sequence
  siRNA sequence
aggaucgcagggagaggcugu

SEQ ID NO: 84 - RNA, 21 nt, Artificial Sequence
  siRNA sequence
acagccucucccugcgauccu

SEQ ID NO: 85 - RNA, 21 nt, Artificial Sequence
  siRNA sequence
ggaucgcagggagaggcugua

SEQ ID NO: 86 - RNA, 21 nt, Artificial Sequence
  siRNA sequence
uacagccucucccugcgaucc

SEQ ID NO: 87 - RNA, 21 nt, Artificial Sequence
  siRNA sequence
gaucgcagggagaggcuguau

SEQ ID NO: 88 - RNA, 21 nt, Artificial Sequence
  siRNA sequence
auacagccucucccugcgauc

SEQ ID NO: 89 - DNA, 21 bp, Artificial Sequence
  siRNA sequence
atcgcagggagaggcuguauu

SEQ ID NO: 90 - RNA, 21 nt, Artificial Sequence
  siRNA sequence
aauacagccucucccugcgau

SEQ ID NO: 91 - RNA, 21 nt, Artificial Sequence
  siRNA sequence
ucgcagggagaggcuguauua

SEQ ID NO: 92 - RNA, 21 nt, Artificial Sequence
  siRNA sequence
uaauacagccucucccugcga

SEQ ID NO: 93 - RNA, 21 nt, Artificial Sequence
  siRNA sequence
cgcagggagaggcuguauuaa

SEQ ID NO: 94 - RNA, 21 nt, Artificial Sequence
  siRNA sequence
uuaauacagccucucccugcg

SEQ ID NO: 95 - RNA, 21 nt, Artificial Sequence
  siRNA sequence
gcagggagaggcuguauuaau

SEQ ID NO: 96 - RNA, 21 nt, Artificial Sequence
  siRNA sequence
auuaauacagccucucccugc

SEQ ID NO: 97 - RNA, 21 nt, Artificial Sequence
  siRNA sequence
cagggagaggcuguauuaauu

SEQ ID NO: 98 - RNA, 21 nt, Artificial Sequence
  siRNA sequence
aauuaauacagccucucccug

SEQ ID NO: 99 - RNA, 21 nt, Artificial Sequence
  siRNA sequence
agggagaggcuguauuaauua

SEQ ID NO: 100 - RNA, 21 nt, Artificial Sequence
  siRNA sequence
uaauuaauacagccucucccu

SEQ ID NO: 101 - RNA, 21 nt, Artificial Sequence
  siRNA sequence
gggagaggcuguauuaauuag

SEQ ID NO: 102 - RNA, 21 nt, Artificial Sequence
  siRNA sequence
cuaauuaauacagccucuccc

SEQ ID NO: 103 - RNA, 21 nt, Artificial Sequence
  siRNA sequence
ggagaggcuguauuaauuagc

SEQ ID NO: 104 - RNA, 21 nt, Artificial Sequence
  siRNA sequence
gcuaauuaauacagccucucc

SEQ ID NO: 105 - RNA, 21 nt, Artificial Sequence
  siRNA sequence
gagaggcuguauuaauuagcc

SEQ ID NO: 106 - RNA, 21 nt, Artificial Sequence
  siRNA sequence
ggcuaauuaauacagccucuc

SEQ ID NO: 107 - RNA, 21 nt, Artificial Sequence
  siRNA sequence
agaggcuguauuaauuagccu

SEQ ID NO: 108 - RNA, 21 nt, Artificial Sequence
  siRNA sequence
aggcuaauuaauacagccucu

SEQ ID NO: 109 - RNA, 21 nt, Artificial Sequence
  siRNA sequence
gaggcuguauuaauuagccug

SEQ ID NO: 110 - RNA, 21 nt, Artificial Sequence
  siRNA sequence
caggcuaauuaauacagccuc

SEQ ID NO: 111 - RNA, 21 nt, Artificial Sequence
  siRNA sequence
aggcuguauuaauuagccugg

SEQ ID NO: 112 - RNA, 21 nt, Artificial Sequence
  siRNA sequence
ccaggcuaauuaauacagccu

SEQ ID NO: 113 - RNA, 21 nt, Artificial Sequence
  siRNA sequence
ggcuguauuaauuagccuggc

SEQ ID NO: 114 - RNA, 21 nt, Artificial Sequence
  siRNA sequence
gccaggcuaauuaauacagcc

SEQ ID NO: 115 - RNA, 21 nt, Artificial Sequence
  siRNA sequence
gcuguauuaauuagccuggcu

SEQ ID NO: 116 - RNA, 21 nt, Artificial Sequence
  siRNA sequence
agccaggcuaauuaauacagc

SEQ ID NO: 117 - RNA, 21 nt, Artificial Sequence
  siRNA sequence
cuguauuaauuagccuggcuc

SEQ ID NO: 118 - RNA, 21 nt, Artificial Sequence
  siRNA sequence
gagccaggcuaauuaauacag

SEQ ID NO: 119 - RNA, 21 nt, Artificial Sequence
  siRNA sequence
uguauuaauuagccuggcucc

SEQ ID NO: 120 - RNA, 21 nt, Artificial Sequence
  siRNA sequence
ggagccaggcuaauuaauaca

SEQ ID NO: 121 - DNA, 21 bp, Artificial Sequence
  siRNA sequence
  modified_base (20)..(21): 2′-deoxy-nucleotide
aggaucgcagggagaggcutt

SEQ ID NO: 122 - DNA, 21 bp, Artificial Sequence
  siRNA sequence
  modified_base (20)..(21): 2′-deoxy-nucleotide
agccucucccugcgauccutt

SEQ ID NO: 123 - DNA, 21 bp, Artificial Sequence
  sirRNA sequence
  modified_base (20)..(21): 2′-deoxy-nucleotide
ggaucgcagggagaggcugtt

SEQ ID NO: 124 - DNA, 21 bp, Artificial Sequence
  siRNA sequence
  modified_base (20)..(21): 2′-deoxy-nucleotide
cagccucucccugcgaucctt

SEQ ID NO: 125 - DNA, 21 bp, Artificial Sequence
  siRNA sequence
  modified_base (20)..(21): 2′-deoxy-nucleotide
gaucgcagggagaggcugutt

SEQ ID NO: 126 - DNA, 21 bp, Artificial Sequence
  siRNA sequence
  modified_base (20)..(21): 2′-deoxy-nucleotide
acagccucucccugcgauctt

SEQ ID NO: 127 - DNA, 21 bp, Artificial Sequence
  siRNA sequence
  modified_base (20)..(21): 2′-deoxy-nucleotide
aucgcagggagaggcuguatt

SEQ ID NO: 128 - DNA, 21 bp, Artificial Sequence
  siRNA sequence
  modified_base (20)..(21): 2′-deoxy-nucleotide
uacagccucucccugcgautt

SEQ ID NO: 129 - DNA, 21 bp, Artificial Sequence
  siRNA sequence
  modified_base (20)..(21): 2′-deoxy-nucleotide
ucgcagggagaggcuguautt

SEQ ID NO: 130 - DNA, 21 bp, Artificial Sequence
  siRNA sequence
  modified_base (20)..(21): 2′-deoxy-nucleotide
auacagccucucccugcgatt

SEQ ID NO: 131 - DNA, 21 bp, Artificial Sequence
  siRNA sequence
  modified_base (20)..(21): 2′-deoxy-nucleotide
cgcagggagaggcuguauutt

SEQ ID NO: 132 - DNA, 21 bp, Artificial Sequence
  siRNA sequence
  modified_base (20)..(21): 2′-deoxy-nucleotide
aauacagccucucccugcgtt

SEQ ID NO: 133 - DNA, 21 bp, Artificial Sequence
  siRNA sequence
  modified_base (20)..(21): 2′-deoxy-nucleotide
gcagggagaggcuguauuatt

SEQ ID NO: 134 - DNA, 21 bp, Artificial Sequence
  siRNA sequence
  modified_base (20)..(21): 2′-deoxy-nucleotide
uaauacagccucucccugctt

SEQ ID NO: 135 - DNA, 21 bp, Artificial Sequence
  siRNA sequence
  modified_base (20)..(21): 2′-deoxy-nucleotide
cagggagaggcuguauuaatt

SEQ ID NO: 136 - DNA, 21 bp, Artificial Sequence
  siRNA sequence
  modified_base (20)..(21): 2′-deoxy-nucleotide
uuaauacagccucucccugtt

SEQ ID NO: 137 - DNA, 21 bp, Artificial Sequence
  siRNA sequence
  modified_base (20)..(21): 2′-deoxy-nucleotide
agggagaggcuguauuaautt

SEQ ID NO: 138 - DNA, 21 bp, Artificial Sequence
  siRNA sequence
  modified_base (20)..(21): 2′-deoxy-nucleotide
auuaauacagccucucccutt

SEQ ID NO: 139 - DNA, 21 bp, Artificial Sequence
  siRNA sequence
  modified_base (20)..(21): 2′-deoxy-nucleotide
gggagaggcuguauuaauutt

SEQ ID NO: 140 - DNA, 21 bp, Artificial Sequence
  siRNA sequence
  modified_base (20)..(21): 2′-deoxy-nucleotide
aauuaauacagccucuccctt

SEQ ID NO: 141 - DNA, 21 bp, Artificial Sequence
  siRNA sequence
  modified_base (20)..(21): 2′-deoxy-nucleotide
ggagaggcuguauuaauuatt

SEQ ID NO: 142 - DNA, 21 bp, Artificial Sequence
  siRNA sequence
  modified_base (20)..(21): 2′-deoxy-nucleotide
uaauuaauacagccucucctt

SEQ ID NO: 143 - DNA, 21 bp, Artificial Sequence
  siRNA sequence
  modified_base (20)..(21): 2′-deoxy-nucleotide
gagaggcuguauuaauuagtt

SEQ ID NO: 144 - DNA, 21 bp, Artificial Sequence
  siRNA sequence
  modified_base (20)..(21): 2′-deoxy-nucleotide
cuaauuaauacagccucuctt

SEQ ID NO: 145 - DNA, 21 bp, Artificial Sequence
  siRNA sequence
  modified_base (20)..(21): 2′-deoxy-nucleotide
agaggcuguauuaauuagctt

SEQ ID NO: 146 - DNA, 21 bp, Artificial Sequence
  siRNA sequence
  modified_base (20)..(21): 2′-deoxy-nucleotide
gcuaauuaauacagccucutt

SEQ ID NO: 147 - DNA, 21 bp, Artificial Sequence
  siRNA sequence
  modified_base (20)..(21): 2′-deoxy-nucleotide
gaggcuguauuaauuagcctt

SEQ ID NO: 148 - DNA, 21 bp, Artificial Sequence
  siRNA sequence
  modified_base (20)..(21): 2′-deoxy-nucleotide
ggcuaauuaauacagccuctt

SEQ ID NO: 149 - DNA, 21 bp, Artificial Sequence
  siRNA sequence
  modified_base (20)..(21): 2′-deoxy-nucleotide
aggcuguauuaauuagccutt

SEQ ID NO: 150 - DNA, 21 bp, Artificial Sequence
  siRNA sequence
  modified_base (20)..(21): 2′-deoxy-nucleotide
aggcuaauuaauacagccutt

SEQ ID NO: 151 - DNA, 21 bp, Artificial Sequence
  siRNA sequence
  modified_base (20)..(21): 2′-deoxy-nucleotide
ggcuguauuaauuagccugtt

SEQ ID NO: 152 - DNA, 21 bp, Artificial Sequence
  siRNA sequence
  modified_base (20)..(21): 2′-deoxy-nucleotide
caggcuaauuaauacagcctt

SEQ ID NO: 153 - DNA, 21 bp, Artificial Sequence
  siRNA sequence
  modified_base (20)..(21): 2′-deoxy-nucleotide
gcuguauuaauuagccuggtt

SEQ ID NO: 154 - DNA, 21 bp, Artificial Sequence
  siRNA sequence
  modified_base (20)..(21): 2′-deoxy-nucleotide
ccaggcuaauuaauacagctt

SEQ ID NO: 155 - DNA, 21 bp, Artificial Sequence
  siRNA sequence
  modified_base (20)..(21): 2′-deoxy-nucleotide
cuguauuaauuagccuggctt

SEQ ID NO: 156 - DNA, 21 bp, Artificial Sequence
  siRNA sequence
  modified_base (20)..(21): 2′-deoxy-nucleotide
gccaggcuaauuaauacagtt

SEQ ID NO: 157 - DNA, 21 bp, Artificial Sequence
  siRNA sequence
  modified_base (20)..(21): 2′-deoxy-nucleotide
uguauuaauuagccuggcutt

SEQ ID NO: 158 - DNA, 21 bp, Artificial Sequence
  siRNA sequence
  modified_base (20)..(21): 2′-deoxy-nucleotide
agccaggcuaauuaauacatt

SEQ ID NO: 159 - DNA, 22 bp, Artificial Sequence
  siRNA sequence
  modified_base (21)..(22): 2′-deoxy-nucleotide
caggaucgcagggagaggcutt

SEQ ID NO: 160 - DNA, 22 bp, Artificial Sequence
  siRNA sequence
  modified_base (21)..(22): 2′-deoxy-nucleotide
agccucucccugcgauccugtt

SEQ ID NO: 161 - DNA, 22 bp, Artificial Sequence
  siRNA sequence
  modified_base (21)..(22): 2′-deoxy-nucleotide
aggaucgcagggagaggcugtt

SEQ ID NO: 162 - DNA, 22 bp, Artificial Sequence
  siRNA sequence
  modified_base (21)..(22): 2′-deoxy-nucleotide
cagccucucccugcgauccutt

SEQ ID NO: 163 - DNA, 22 bp, Artificial Sequence
  siRNA sequence
  modified_base (21)..(22): 2′-deoxy-nucleotide
ggaucgcagggagaggcugutt

SEQ ID NO: 164 - DNA, 22 bp, Artificial Sequence
  siRNA sequence
  modified_base (21)..(22): 2′-deoxy-nucleotide
acagccucucccugcgaucctt

SEQ ID NO: 165 - DNA, 22 bp, Artificial Sequence
  siRNA sequence
  modified_base (21)..(22): 2′-deoxy-nucleotide
gaucgcagggagaggcuguatt

SEQ ID NO: 166 - DNA, 22 bp, Artificial Sequence
  siRNA sequence
  modified_base (21)..(22): 2′-deoxy-nucleotide
uacagccucucccugcgauctt

SEQ ID NO: 167 - DNA, 22 bp, Artificial Sequence
  siRNA sequence
  modified_base (21)..(22): 2′-deoxy-nucleotide
aucgcagggagaggcuguautt

SEQ ID NO: 168 - DNA, 22 bp, Artificial Sequence
  siRNA sequence
  modified_base (21)..(22): 2′-deoxy-nucleotide
auacagccucucccugcgautt

SEQ ID NO: 169 - DNA, 22 bp, Artificial Sequence
  siRNA sequence
  modified_sequence (21)..(22): 2′-deoxy-nucleotide
ucgcagggagaggcuguauutt

SEQ ID NO: 170 - DNA, 22 bp, Artificial Sequence
  siRNA sequence
  modified_base (21)..(22): 2′-deoxy-nucleotide
aauacagccucucccugcgatt

SEQ ID NO: 171 - DNA, 22 bp, Artificial Sequence
  siRNA sequence
  modified_base (21)..(22): 2′-deoxy-nucleotide
cgcagggagaggcuguauuatt

SEQ ID NO: 172 - DNA, 22 bp, Artificial Sequence
  siRNA sequence
  modified_base (21)..(22): 2′-deoxy-nucleotide
uaauacagccucucccugcgtt

SEQ ID NO: 173 - DNA, 22 bp, Artificial Sequence
  siRNA sequence
  modified_base (21)..(22): 2′-deoxy-nucleotide
gcagggagaggcuguauuaatt

SEQ ID NO: 174 - DNA, 22 bp, Artificial Sequence
  siRNA sequence
  modified_base (21)..(22): 2′-deoxy-nucleotide
uuaauacagccucucccugctt

SEQ ID NO: 175 - DNA, 22 bp, Artificial Sequence
  siRNA sequence
  modified_base (21)..(22): 2′-deoxy-nucleotide
cagggagaggcuguauuaautt

SEQ ID NO: 176 - DNA, 22 bp, Artificial Sequence
  siRNA sequence
  modified_base (21)..(22): 2′-deoxy-nucleotide
auuaauacagccucucccugtt

SEQ ID NO: 177 - DNA, 22 bp, Artificial Sequence
  siRNA sequence
  modified_base (21)..(22): 2′-deoxy-nucleotide
agggagaggcuguauuaauutt

SEQ ID NO: 178 - DNA, 22 bp, Artificial Sequence
  siRNA sequence
  modified_base (21)..(22): 2′-deoxy-nucleotide
aauuaauacagccucucccutt

SEQ ID NO: 179 - DNA, 22 bp, Artificial Sequence
  siRNA sequence
  modified_base (21)..(22): 2′-deoxy-nucleotide
gggagaggcuguauuaauuatt

SEQ ID NO: 180 - DNA, 22 bp, Artificial Sequence
  siRNA sequence
  modified_base (21)..(22): 2′-deoxy-nucleotide
uaauuaauacagccucuccctt

SEQ ID NO: 181 - DNA, 22 bp, Artificial Sequence
  siRNA sequence
  modified_base (21)..(22): 2′-deoxy-nucleotide
ggagaggcuguauuaauuagtt

SEQ ID NO: 182 - DNA, 22 bp, Artificial Sequence
  siRNA sequence
  modified_base (21)..(22): 2′-deoxy-nucleotide
cuaauuaauacagccucucctt

SEQ ID NO: 183 - DNA, 22 bp, Artificial Sequence
  siRNA sequence
  modified_base (21)..(22): 2′-deoxy-nucleotide
gagaggcuguauuaauuagctt

SEQ ID NO: 184 - DNA, 22 bp, Artificial Sequence
  siRNA sequence
  modified_base (21)..(22): 2′-deoxy-nucleotide
gcuaauuaauacagccucuctt

SEQ ID NO: 185 - DNA, 22 bp, Artificial Sequence
  siRNA sequence
  modified_base (21)..(22): 2′-deoxy-nucleotide
agaggcuguauuaauuagcctt

SEQ ID NO: 186 - DNA, 22 bp, Artificial Sequence
  siRNA sequence
  modified_base (21)..(22): 2′-deoxy-nucleotide
ggcuaauuaauacagccucutt

SEQ ID NO: 187 - DNA, 22 bp, Artificial Sequence
  siRNA sequence
  modified_base (21)..(22): 2′-deoxy-nucleotide
gaggcuguauuaauuagccutt

SEQ ID NO: 188 - DNA, 22 bp, Artificial Sequence
  siRNA sequence
  modified_base (21)..(22): 2′-deoxy-nucleotide
aggcuaauuaauacagccuctt

SEQ ID NO: 189 - DNA, 22 bp, Artificial Sequence
  siRNA sequence
  modified_base (21)..(22): 2′-deoxy-nucleotide
aggcuguauuaauuagccugtt

SEQ ID NO: 190 - DNA, 22 bp, Artificial Sequence
  siRNA sequence
  modified_base (21)..(22): 2′-deoxy-nucleotide
caggcuaauuaauacagccutt

SEQ ID NO: 191 - DNA, 22 bp, Artificial Sequence
  siRNA sequence
  modified_base (21)..(22): 2′-deoxy-nucleotide
ggcuguauuaauuagccuggtt

SEQ ID NO: 192 - DNA, 22 bp, Artificial Sequence
  siRNA sequence
  modified_base (21)..(22): 2′-deoxy-nucleotide
ccaggcuaauuaauacagcctt

SEQ ID NO: 193 - DNA, 22 bp, Artificial Sequence
  siRNA sequence
  modified_base (21)..(22): 2′-deoxy-nucleotide
gcuguauuaauuagccuggctt

SEQ ID NO: 194 - DNA, 22 bp, Artificial Sequence
  siRNA sequence
  modified_base (21)..(22): 2′-deoxy-nucleotide
gccaggcuaauuaauacagctt

SEQ ID NO: 195 - DNA, 22 bp, Artificial Sequence
  siRNA sequence
  modified_base (21)..(22): 2′-deoxy-nucleotide
cuguauuaauuagccuggcutt

SEQ ID NO: 196 - DNA, 22 bp, Artificial Sequence
  siRNA sequence
  modified_base (21)..(22): 2′-deoxy-nucleotide
agccaggcuaauuaauacagtt

SEQ ID NO: 197 - DNA, 22 bp, Artificial Sequence
  siRNA sequence
  modified_base (21)..(22): 2′-deoxy-nucleotide
uguauuaauuagccuggcuctt

SEQ ID NO: 198 - DNA, 22 bp, Artificial Sequence
  siRNA sequence
  modified_base (21)..(22): 2′-deoxy-nucleotide
gagccaggcuaauuaauacatt

SEQ ID NO: 199 - DNA, 23 bp, Artificial Sequence
  siRNA sequence
  modified_base (22)..(23): 2′-deoxy-nucleotide
acaggaucgcagggagaggcutt

SEQ ID NO: 200 - DNA, 23 bp, Artificial Sequence
  siRNA sequence
  modified_base (22)..(23): 2′-deoxy-nucleotide
agccucucccugcgauccugutt

SEQ ID NO: 201 - DNA, 23 bp, Artificial Sequence
  siRNA sequence
  modified_base (22)..(23): 2′-deoxy-nucleotide
caggaucgcagggagaggcugtt

SEQ ID NO: 202 - DNA, 23 bp, Artificial Sequence
  siRNA sequence
  modified_base (22)..(23): 2′-deoxy-nucleotide
cagccucucccugcgauccugtt

SEQ ID NO: 203 - DNA, 23 bp, Artificial Sequence
  siRNA sequence
  modified_base (22)..(23): 2′-deoxy-nucleotide
aggaucgcagggagaggcugutt

SEQ ID NO: 204 - DNA, 23 bp, Artificial Sequence
  siRNA sequence
  modiied_base (22)..(23): 2′-deoxy-nucleotide
acagccucucccugcgauccutt

SEQ ID NO: 205 - DNA, 23 bp, Artificial Sequence
  siRNA sequence
  modified_base (22)..(23): 2′-deoxy-nucleotide
ggaucgcagggagaggcuguatt

SEQ ID NO: 206 - DNA, 23 bp, Artificial Sequence
  siRNA sequence
  modified_base (22)..(23): 2′-deoxy-nucleotide
uacagccucucccugcgaucctt

SEQ ID NO: 207 - DNA, 23 bp, Artificial Sequence
  siRNA sequence
  modified_base (22)..(23): 2′-deoxy-nucleotide
gaucgcagggagaggcuguautt

SEQ ID NO: 208 - DNA, 23 bp, Artificial Sequence
  siRNA sequence
  modified_base (22)..(23): 2′-deoxy-nucleotide
auacagccucucccugcgauctt

SEQ ID NO: 209 - DNA, 23 bp, Artificial Sequence
  siRNA sequence
  modified_base (22)..(23): 2′-deoxy-nucleotide
atcgcagggagaggcuguauutt

SEQ ID NO: 210 - DNA, 23 bp, Artificial Sequence
  siRNA sequence
  modified_base (22)..(23): 2′-deoxy-nucleotide
aauacagccucucccugcgautt

SEQ ID NO: 211 - DNA, 23 bp, Artificial Sequence
  siRNA sequence
  modified_base (22)..(23): 2′-deoxy-nucleotide
ucgcagggagaggcuguauuatt

SEQ ID NO: 212 - DNA, 23 bp, Artificial Sequence
  siRNA sequence
  modified_base (22)..(23): 2′-deoxy-nucleotide
uaauacagccucucccugcgatt

SEQ ID NO: 213 - DNA, 23 bp, Artificial Sequence
  siRNA sequence
  modified_base (22)..(23): 2′-deoxy-nucleotide
cgcagggagaggcuguauuaatt

SEQ ID NO: 214 - DNA, 23 bp, Artificial Sequence
  siRNA sequence
  modified_base (22)..(23): 2′-deoxy-nucleotide
uuaauacagccucucccugcgtt

SEQ ID NO: 215 - DNA, 23 bp, Artificial Sequence
  siRNA sequence
  modified_base (22)..(23): 2′-deoxy-nucleotide
gcagggagaggcuguauuaautt

SEQ ID NO: 216 - DNA, 23 bp, Artificial Sequence
  siRNA sequence
  modified_base (22)..(23): 2′-deoxy-nucleotide
auuaauacagccucucccugctt

SEQ ID NO: 217 - DNA, 23 bp, Artificial Sequence
  siRNA sequence
  modified_base (22)..(23): 2′-deoxy-nucleotide
cagggagaggcuguauuaauutt

SEQ ID NO: 218 - DNA, 23 bp, Artificial Sequence
  siRNA sequence
  modified_base (22)..(23): 2′-deoxy-nucleotide
aauuaauacagccucucccugtt

SEQ ID NO: 219 - DNA, 23 bp, Artificial Sequence
  siRNA sequence
  modified_base (22)..(23): 2′-deoxy-nucleotide
agggagaggcuguauuaauuatt

SEQ ID NO: 220 - DNA, 23 bp, Artificial Sequence
  siRNA sequence
  modified_base (22)..(23): 2′-deoxy-nucleotide
uaauuaauacagccucucccutt

SEQ ID NO: 221 - DNA, 23 bp, Artificial Sequence
  siRNA sequence
  modified_base (22)..(23): 2′-deoxy-nucleotide
gggagaggcuguauuaauuagtt

SEQ ID NO: 222 - DNA, 23 bp, Artificial Sequence
  siRNA sequence
  modified_base (22)..(23): 2′-deoxy-nucleotide
cuaauuaauacagccucuccctt

SEQ ID NO: 223 - DNA, 23 bp, Artificial Sequence
  siRNA sequence
  modified_base (22)..(23): 2′-deoxy-nucleotide
ggagaggcuguauuaauuagctt

SEQ ID NO: 224 - DNA, 23 bp, Artificial Sequence
  siRNA sequence
  modified_base (22)..(23): 2′-deoxy-nucleotide
gcuaauuaauacagccucucctt

SEQ ID NO: 225 - DNA, 23 bp, Artificial Sequence
  siRNA sequence
  modified_base (22)..(23): 2′-deoxy-nucleotide
gagaggcuguauuaauuagcctt

SEQ ID NO: 226 - DNA, 23 bp, Artificial Sequence
  siRNA sequence
  modified_base (22)..(23): 2′-deoxy-nucleotide
ggcuaauuaauacagccucuctt

SEQ ID NO: 227 - DNA, 23 bp, Artificial Sequence
  siRNA sequence
  modified_base (22)..(23): 2′-deoxy-nucleotide
agaggcuguauuaauuagccutt

SEQ ID NO: 228 - DNA, 23 bp, Artificial Sequence
  siRNA sequence
  modified_base (22)..(23): 2′-deoxy-nucleotide
aggcuaauuaauacagccucutt

SEQ ID NO: 229 - DNA, 23 bp, Artificial Sequence
  siRNA sequence
  modified_base (22)..(23): 2′-deoxy-nucleotide
gaggcuguauuaauuagccugtt

SEQ ID NO: 230 - DNA, 23 bp, Artificial Sequence
  siRNA sequence
  modified_base (22)..(23): 2′-deoxy-nucleotide
caggcuaauuaauacagccuctt

SEQ ID NO: 231 - DNA, 23 bp, Artificial Sequence
  siRNA sequence
  modified_base (22)..(23): 2′-deoxy-nucleotide
aggcuguauuaauuagccuggtt

SEQ ID NO: 232 - DNA, 23 bp, Artificial Sequence
  siRNA sequence
  modified_base (22)..(23): 2′-deoxy-nucleotide
ccaggcuaauuaauacagccutt

SEQ ID NO: 233 - DNA, 23 bp, Artificial Sequence
  siRNA sequence
  modified_base (22)..(23): 2′-deoxy-nucleotide
ggcuguauuaauuagccuggctt

SEQ ID NO: 234 - DNA, 23 bp, Artificial Sequence
  siRNA sequence
  modified_base (22)..(23): 2′-deoxy-nucleotide
gccaggcuaauuaauacagcctt

SEQ ID NO: 235 - DNA, 23 bp, Artificial Sequence
  siRNA sequence
  modified_base (22)..(23): 2′-deoxy-nucleotide
gcuguauuaauuagccuggcutt

SEQ ID NO: 236 - DNA, 23 bp, Artificial Sequence
  siRNA sequence
  modified_base (22)..(23): 2′-deoxy-nucleotide
agccaggcuaauuaauacagctt

SEQ ID NO: 237 - DNA, 23 bp, Artificial Sequence
  siRNA sequence
  modified_base (22)..(23): 2′-deoxy-nucleotide
cuguauuaauuagccuggcuctt

SEQ ID NO: 238 - DNA, 23 bp, Artificial Sequence
  siRNA sequence
  modified_base (22)..(23): 2′-deoxy-nucleotide
gagccaggcuaauuaauacagtt

SEQ ID NO: 239 - DNA, 23 bp, Artificial Sequence
  siRNA sequence
  modified_base (22)..(23): 2′-deoxy-nucleotide
uguauuaauuagccuggcucctt

SEQ ID NO: 240 - DNA, 23 bp, Artificial Sequence
  siRNA sequence
  modified_base (22)..(23): 2′-deoxy-nucleotide
ggagccaggcuaauuaauacatt

SEQ ID NO: 241 - RNA, 19 nt, Artificial Sequence
  siRNA sequence
uagcgacuaaacacaucaa

SEQ ID NO: 242 - RNA, 19 nt, Artificial Sequence
  siRNA sequence
ccuacggacagcagaguua

SEQ ID NO: 243 - RNA, 19 nt, Artificial Sequence
  siRNA sequence
gauuauacccaacaagcaa

SEQ ID NO: 244 - RNA, 19 nt, Artificial Sequence
  siRNA sequence
gaucaauccuccaugagua

SEQ ID NO: 245 - RNA, 19 nt, Artificial Sequence
  siRNA sequence
cgggacagcccaugauuaa

SEQ ID NO: 246 - DNA, 1759 bp, Homo sapiens
tgcgcggacatggcctcaaacgattatacccaacaagcaacccaaagctatggggcctaccccacccagcccgggcagggctattcccagcagagcagtcagccctacggacagcagagttacagtggttatagccagtccacggacacttcaggctatggccagagcagctattcttcttatggccagagccagaacagctatggaactcagtcaactccccagggatatggctcgactggcggctatggcagtagccagagctcccaatcgtcttacgggcagcagtcctcctatcctggctatggccagcagccagctcccagcagcacctcgggaagttacggtagcagttctcagagcagcagctatgggcagccccagagtgggagctacagccagcagcctagctatggtggacagcagcaaagctatggacagcagcaaagctataatccccctcagggctatggacagcagaaccagtacaacagcagcagtggtggtggaggtggaggtggaggtggaggtaactatggccaagatcaatcctccatgagtagtggtggtggcagtggtggcggttatggcaatcaagaccagagtggtggaggtggcagcggtggctatggacagcaggaccgtggaggccgcggcaggggtggcagtggtggcggcggcggcggcggcggtggtggttacaaccgcagcagtggtggctatgaacccagaggtcgtggaggtggccgtggaggcagaggtggcatgggcggaagtgaccgtggtggcttcaataaatttggtggccctcgggaccaaggatcacgtcatgactccgaacaggataattcagacaacaacaccatctttgtgcaaggcctgggtgagaatgttacaattgagtctgtggctgattacttcaagcagattggtattattaagacaaacaagaaaacgggacagcccatgattaatttgtacacagacagggaaactggcaagctgaagggagaggcaacggtctcttttgatgacccaccttcagctaaagcagctattgactggtttgatggtaaagaattctccggaaatcctatcaaggtctcatttgctactcgccgggcagactttaatcggggtggtggcaatggtcgtggaggccgagggcgaggaggacccatgggccgtggaggctatggaggtggtggcagtggtggtggtggccgaggaggatttcccagtggaggtggtggcggtggaggacagcagcgagctggtgactggaagtgtcctaatcccacctgtgagaatatgaacttctcttggaggaatgaatgcaaccagtgtaaggcccctaaaccagatggcccaggagggggaccaggtggctctcacatggggggtaactacggggatgatcgtcgtggtggcagaggaggctatgatcgaggcggctaccggggccgcggcggggaccgtggaggcttccgagggggccggggtggtggggacagaggtggctttggccctggcaagatggattccaggggtgagcacagacaggatcgcagggagaggccgtattaattagcctggctccccaggttctggaacagctttttgtcctgtacccagtgttaccctcgttattttgtaaccttccaattcctgatcacccaagggtttttttgtgtcggactatgtaattgtaactatacctctggttcccattaaaagtgaccattttagttaaaaaaaa

SEQ ID NO: 247 - DNA, 1759 bp, Homo sapiens
tgcgcggacatggcctcaaacgattatacccaacaagcaacccaaagctatggggcctaccccacccagcccgggcagggctattcccagcagagcagtcagccctacggacagcagagttacagtggttatagccagtccacggacacttcaggctatggccagagcagctattcttcttatggccagagccagaacagctatggaactcagtcaactccccagggatatggctcgactggcggctatggcagtagccagagctcccaatcgtcttacgggcagcagtcctcctatcctggctatggccagcagccagctcccagcagcacctcgggaagttacggtagcagttctcagagcagcagctatgggcagccccagagtgggagctacagccagcagcctagctatggtggacagcagcaaagctatggacagcagcaaagctataatccccctcagggctatggacagcagaaccagtacaacagcagcagtggtggtggaggtggaggtggaggtggaggtaactatggccaagatcaatcctccatgagtagtggtggtggcagtggtggcggttatggcaatcaagaccagagtggtggaggtggcagcggtggctatggacagcaggaccgtggaggccgcggcaggggtggcagtggtggcggcggcggcggcggcggtggtggttacaaccgcagcagtggtggctatgaacccagaggtcgtggaggtggccgtggaggcagaggtggcatgggcggaagtgaccgtggtggcttcaataaatttggtggccctcgggaccaaggatcacgtcatgactccgaacaggataattcagacaacaacaccatctttgtgcaaggcctgggtgagaatgttacaattgagtctgtggctgattacttcaagcagattggtattattaagacaaacaagaaaacgggacagcccatgattaatttgtacacagacagggaaactggcaagctgaagggagaggcaacggtctcttttgatgacccaccttcagctaaagcagctattgactggtttgatggtaaagaattctccggaaatcctatcaaggtctcatttgctactcgccgggcagactttaatcggggtggtggcaatggtcgtggaggccgagggcgaggaggacccatgggccgtggaggctatggaggtggtggcagtggtggtggtggccgaggaggatttcccagtggaggtggtggcggtggaggacagcagcgagctggtgactggaagtgtcctaatcccacctgtgagaatatgaacttctcttggaggaatgaatgcaaccagtgtaaggcccctaaaccagatggcccaggagggggaccaggtggctctcacatggggggtaactacggggatgatcgtcgtggtggcagaggaggctatgatcgaggcggctaccggggccgcggcggggaccgtggaggcttccgagggggccggggtggtggggacagaggtggctttggccctggcaagatggattccaggggtgagcacagacaggatcgcagggagaggctgtattaattagcctggctccccaggttctggaacagctttttgtcctgtacccagtgttaccctcgttattttgtaaccttccaattcctgatcacccaagggtttttttgtgtcggactatgtaattgtaactatacctctggttcccattaaaagtgaccattttagttaaaaaaaa

SEQ ID NO: 248 - DNA, 25 bp, Artificial Sequence
  Primer
tctcgagctcaagcttcgaattctg

SEQ ID NO: 249 - DNA, 25 bp, Artificial Sequence
  Primer
tctttcttcaccggcatctgcatcc

SEQ ID NO: 250 - DNA, 35 bp, Artificial Sequence
  Primer
gccggtgaagaaagaatggcctcaaacgattatac

SEQ ID NO: 251 - DNA, 38 bp, Artificial Sequence
  Primer
agcttgagctcgagattttttttaactaaaatggtcac

SEQ ID NO: 252 - DNA, 25 bp, Artificial Sequence
  Primer
tctcgagctcaagcttcgaattctg

SEQ ID NO: 253 - DNA, 25 bp, Artificial Sequence
  siRNA sequence
tctgagtccggagctgtgccccagt

SEQ ID NO: 254 - DNA, 35 bp, Artificial Sequence
  Primer
agctccggactcagaatggcctcaaacgattatac

SEQ ID NO: 255 - DNA, 38 bp, Artificial Sequence; identical to SEQ ID NO: 251
  Primer

SEQ ID NO: 256 - DNA, 30 bp, Artificial Sequence
  Primer
attaggcgcgccaccatggagagcgacgag

SEQ ID NO: 257 - DNA, 23 bp, Artificial Sequence
  Primer
taatgcggccgcttgagctcgag

SEQ ID NO: 258 - DNA, 32 bp, Artificial Squence
  Primer
attaggcgcgccaccatggtgggtgaggatag

SEQ ID NO: 259 - DNA, 23 bp, Artificial Sequence
  Primer
taatgcggccgcttgagctcgag

SEQ ID NO: 260 - DNA, 18 bp, Artificial Sequence
  Primer
caccaactgggacgacat

SEQ ID NO: 261 - DNA, 18 bp, Artificial Sequence
  Primer
acagcctggatagcaacg

SEQ ID NO: 262 - DNA, 21 bp, Artificial Sequence
  Primer
cttatggccagagccagaaca

SEQ ID NO: 263 - DNA, 20 bp, Artificial Sequence
  Primer
atcatgggctgtcccgtttt

SEQ ID NO: 264 - DNA, 19 bp, Artificial Sequence
  Primer
tgggcatcgtggagtacca

SEQ ID NO: 265 - DNA, 24 bp, Artificial Sequence
  Primer
tgcttgttgggtataatcgtttga

SEQ ID NO: 266 - DNA, 20 bp, Artificial Sequence
  Primer
ctgcgacctgcctagcaaac

SEQ ID NO: 267 - DNA, 22 bp, Artificial Sequence
  Primer
ccatagctttgggttgcttgtt

SEQ ID NO: 268 - DNA, 15 bp, Artificial Sequence
  Probe
agaccccggatgcag

SEQ ID NO: 269 - DNA, 15 bp, Artificial Sequence
  Probe
acagctccggactca